(19) United States
(12) Patent Application Publication
(10) Pub. No.: US 2024/0115509 A1
(43) Pub. Date: Apr. 11, 2024

(54) A PROBIOTIC GRANULE HAVING A UNIFIED STABILIZING COATING AND A METHOD FOR THE PRODUCTION THEREOF

(71) Applicant: AMD PHARMA LTD, Tal Aviv (IL)

(72) Inventors: Adel PENHASI, Modiin (IL); Israel BALUASHVILI, Jersualem (IL)

(21) Appl. No.: 18/276,464

(22) PCT Filed: Feb. 10, 2022
(86) PCT No.: PCT/IL2022/050169

§ 371 Date: Aug. 9, 2023

Related U.S. Application Data
(60) Provisional application No. 63/148,152, filed on Feb. 11, 2021.

(51) Int. Cl. A61K 9/50; A61K 9/00; A61K 35/745; A61K 35/747

(52) U.S. Cl. CPC A61K 9/5036; A61K 9/0056; A61K 9/5015; A61K 9/5089; A61K 35/745; A61K 35/747

(57) ABSTRACT

According to some demonstrative embodiments, there is provided herein a probiotic granule comprising: a core comprising probiotic bacteria; and a single continuous coating layer coating said core comprising at least one an edible hydrophobic solid component such as fat, wax, phospholipid or a fatty acid having a melting point higher than 30° C.; at least one edible water-soluble polymeric stress absorber, dispersed within said hydrophobic solid component.

### FIELD OF THE INVENTION

The present invention is related to the field of probiotics, and particularly to methods and compositions for protecting probiotic bacteria from harmful conditions.

### BACKGROUND OF THE INVENTION

Probiotics are live microbial food supplements which beneficially affect the host by supporting naturally occurring gut flora, by competing with harmful microorganisms in the gastrointestinal tract, by assisting useful metabolic processes and by strengthening the resistance of the host organism against toxic substances.

The beneficial effects that probiotics may induce are numerous. Few examples include the reduction of lactose intolerance, the inhibition of pathogenic bacteria and parasites, the reduction of diarrhea, activity against Helicobacter pylori, the prevention of colon cancer, the improvement or prevention of constipation, the in situ production of vitamins, the modulation of blood fats and the modulation of host immune functions.

In domesticated and aquatic animals probiotics can also improve growth, survival and stress resistance associated with diseases and unfavorable culture conditions.

Therefore, there is considerable interest in the concept of incorporating probiotics into human foodstuffs and into animal feed.

Upon discussing foodstuff which includes probiotics, the probiotic organisms should survive for the lifetime of the product in order to be effective. Probiotic organisms are usually incorporated into milk products, such as yogurts. The need is felt to deliver the beneficial microorganisms in other foodstuff types, for example creams, biscuits fill-in, chocolate, sauces, mayonnaise and etc., especially those which undergo heat treatment during at least one stage of their preparation.

Many probiotics may be temperature, humidity, water activity, and oxygen sensitive and thus suffer from lack of an extended shelf life. Therefore, they need protection during processing, transporting and storage as well as during delivery to the gastro-intestinal tract to maintain viability.

The activity and long-term stability of probiotics bacteria may be affected by a number of environmental factors; for example, temperature, pH, the presence of water/humidity and oxygen or oxidizing or reducing agents. It is well known that, in an aqueous phase, probiotics instantly lose their activity during storage at ambient temperatures (AT), such as room temperature (25 degrees Celsius).

Generally, Probiotic bacteria must be dried before or during mixing with other foodstuff ingredients. The drying process can often result in a significant loss in activity from mechanical, chemical, and osmotic stresses induced by the drying process.

Loss of activity may occur at many distinct stages, including drying during initial manufacturing, food preparation (upon exposure to high temperature, high humidity, oxygen and high pressure), transportation and long-term storage (temperature, oxygen and humid exposure), and after consumption and passage in the gastrointestinal (GI) track (exposure to low pH, proteolytic enzymes and bile salts). Manufacturing food or feedstuffs with live cell organisms or probiotics is particularly challenging, because the probiotics are very sensitive to oxygen, temperature, moisture and high water activity which are in fact the conditions of the foodstuff, feedstuff and supplement products which are packaged using packaging materials with low barrier properties or when they are non-hermetically sealed.

Many probiotics exhibit their beneficial effect mainly when they are alive. Hence, they need to survive the manufacturing process and shelf life of the food. Likewise, they should survive the gastro-intestinal tract conditions such as very low pH existing in stomach, upon consumption of the food before reaching their place of colonization. Although many commercial probiotic products are available for animal and human consumptions, most of them lost their viability during the manufacture process, transport, storage and in the animal/human GI tract.

To compensate for such loss, an excessive quantity of probiotics is often included in the product in anticipation that a portion of the bacteria might survive and reach their target. In addition to questionable shelf-life viability for these products, such practices are certainly not cost-effective.

Various protective agents have been used in the art, with varying degrees of success. These include proteins, certain polymers, skim milk, glycerol, polysaccharides, oligosaccharides and disaccharides. Disaccharides, such as sucrose and trehalose, are particularly attractive cryoprotectants because they actually help plants and microbial cells remain in a state of suspended animation during periods of drought.

Trehalose has been shown to be an effective protectant for a variety of biological materials, both in ambient air-drying and freeze-drying.

Alternatively, the probiotic microorganisms can be encapsulated by enteric coating techniques involve applying a film forming substance, usually by spraying liquids containing enteric polymer and generally other additives such as sugars or proteins onto the dry probiotics (Ko and Ping WO 02/058735). However, the enteric polymers film coating the probiotics upon microencapsulation process usually cannot function as a moisture protecting barrier and generally several layers must be added, to avoid water entering the microcapsules. In addition, such polymers also cannot provide an appropriate protection against oxygen upon very poor oxygen occlusion properties of such polymers.

Giffard and Kendall (US 2005/0079244) disclose a foodstuff in the form of a dried or semi-moist ready-to-eat kibble or powder mix, which contains a combination of a probiotic, prebiotic and a coating of colostrum. Prior to mixing in the food stuff, the probiotic is coated or encapsulated in a polysaccharide, fat, starch, protein or in a sugar matrix using standard encapsulation techniques. Similar to the above disclosure, neither the encapsulating polymers nor the additives used in both core and coating layers have low water vapor and oxygen transmission arte therefore the negative effects of water (humidity) and oxygen cannot be avoided.

Accordingly, it has been proposed to dry sugar-based probiotic systems by foam formation in a very thin layer (Bronshtein WO2005117962), or to use combinations of sugars with a polymeric gelling agent, such as alginate, chitosan, carboxymethylcellulose or carboxyethylcellulose. Cavadini et al. (EP 0 862 863) provide a cereal product comprising a gelatinized starch matrix including a coating or a filling. The probiotic is included with the coating. According to that process, spray-dried probiotics are mixed with a carrier substrate, which may be water, fat or a protein digest. The mixture is then sprayed onto the cereal product and the whole product is dried again. Re-hydrating of the already dried bacteria and the additional coating/drying process is costly and damaging to the bacteria.

US 2005/0019417 A1 describes a method of preparing products containing moisture-sensitive living microorganisms including probiotics, comprising at least the steps through which a suspension of probiotics and a sugar polymer in water miscible solvent is sprayed onto water-soluble, gel-forming solid particles. By these means, the core composed of water soluble gel-forming solid particles may absorb solvent residues and provide protection to probiotics placed onto said core.

Kenneth and Liegh (U.S. Pat. No. 6,900,173) describe the manufacturing of multivitamin protein and probiotic bar for promoting an anabolic state in a person. The dried probiotic bacteria are blended in sugar syrup and several other constituents, and the resultant mixture is then extruded and cut into bars. However, the document does not disclose any process or composition that will improve viability or long-term stability of probiotics in the nutritional bars and there is no indication that the bacteria even survive the process.

US 2004/0175389 (Porubcan) discloses a formulation for protecting probiotic bacteria during passage through the stomach, whilst permitting their release in the intestine. The formulation has also a low water activity and correspondingly long shelf life. The capsule includes a water-free mixture of probiotic bacteria with monovalent alginate salts, and an enteric coating (e.g., gelatin or cellulose encapsulation). Upon contact with acidic environment, the outer shell of the capsule turned into a gel, which provides a protecting barrier against proton influx into the capsule core. However, this composition is only useful for tablets and capsules subjected to storage conditions of very low water activity and further require storage in nitrogen-flushed or vacuum-sealed containers.

WO 03/088755 (Farber and Farber) describes an oral delivery system for functional ingredients uniformly dispersed in a matrix. The matrix components include a sugar, a carbohydrate, a hydrocolloid a polyhydric alcohol and a source of mono- or divalent cations. The delivery system is extruded or molded into a final shape with a moisture content of between 15% and 30% by weight. This type of matrix provides very little protection to the probiotics mostly under refrigerated conditions. No description or direction was provided as to how probiotic bacteria are stabilized during manufacturing or for prolonged storage at room temperatures.

McGrath and Mchale (EP 1382241) describe a method of delivering a microorganism to an animal. The micro-organism is suspended in a matrix of cross-linked alginate and cryopreservant (trehalose or lactose, or a combination of both). The matrix is then freeze or vacuum dried to form dry beads containing live probiotics with a shelf-life stability up to 6 months but only under refrigerated conditions. Here again, no description or direction was provided as to how probiotic bacteria are stabilized during manufacturing or for prolonged storage at room temperatures and high humidity conditions.

Ubbink et al. (US 2005/0153018) disclose the preservation of lactic acid bacteria in moist food. The spray-dried bacteria are added to a composition comprising fats, fermented milk powder and saccharides. That composition is then used as the filling of a confectionary product. The subject matter described in that document avoids the detrimental effects of water by embedding the probiotics in fat or oil rich matrix. However, fat based coating and preserving materials alone do not withstand oxygen and long term exposure to humid conditions.

WO2008076975—Moti Harel and Alicia Bennett disclose a probiotic delivery system that can be consumed as a snack-food or added to a food product. The discloser describes a composition comprises viable probiotic microorganisms preserved in a vacuum dried matrix of sugars, proteins, and polysaccharides. The probiotic remain viable within the tread for a longer time without the need for additional moisture barrier coating.

None of the above compositions provide a mixture that can effectively protect the probiotic in both drying processes and long-term storage at ambient temperatures and varying degrees of humidity. In addition, none of the above compositions provide a mixture that can effectively protect the probitics against oxygen which is a main cause for poor stability for long time in storage conditions causing very limited shelf life. Therefore, there is an urgent need for such a composition that can effectively protect the probiotic bacteria during manufacturing, long-term storage at ambient temperature, humidity and oxygen and during gastrointestinal passage. There is a need also for a preparation process that is cost-effective and capable of entrapping and stabilizing probiotics in the protective mixture with minimal viability loss at the end of the entire operation. There is a need for a protective mixture that provides protection in the stomach while allowing the release of the probiotic along the intestinal tract. There is also a need for a protective mixture that contains only approved ingredients generally regarded as safe (GRAS), and is less costly than those presently being used.

With regard to probiotics, moisture is especially a crucial factor in stability and shelf life of many probiotic bacteria. In many cases the exposure of such probiotics to a certain level of humidity may result in deactivation of bacteria. As a result, the range of food products, especially those with a high level of water activity, in which such bacteria can be incorporated will be limited and the shelf life will be considerably shortened.

Common approaches aimed to limit the damage to the active material, include packaging of the dosage forms containing the moisture sensitive active material in different packaging elements, such as microcapsules, tablets, capsules and the like. However, especially in places where climate is very humid, the special packaging does not provide a complete moisture protection because of the moisture captured inside the above mentioned packaging. Another way to prevent or diminish the damage that may be caused by moisture and to reduce the need for special packaging is to coat the solid dosage forms, containing probiotic bacteria, with materials which have moisture barrier properties.

Such materials have essentially good mechanical properties, especially high flexibility which enables uniform and perfect coating, and also do not affect the basic properties of the dosage forms such as the disintegration time of the dosage form and the release of the probiotics after consumption. However, these materials generally suffer from a low water vapor permeation (WVP) or a low water vapor transition rate (WVTR) especially due to existence of intermolecular micro-voids. Additionally, the coating process is generally carried out using aqueous based-coating solutions causing entrapment of free water between the layers leading to high water content and water activity in the final product which make it inappropriate for highly sensitive probiotic bacteria to water activity. The long coating process accompanied with waste of energy and consequently high production cost, is an additional significant disadvantage of this protection method.

Giovanni and MOGNA, Luca (WO 2013/114185 A1) disclose multilayer microencapsulated lactic bacteria and bifidobacteria; preferably bacteria with probiotic activity using coating material which is selected from the list comprising mono- and di-glycerides of saturated fatty acids, polyglycerols esterified with saturated fatty acids, free saturated fatty acids and glyceryl dipalmitostearate. The main advantages of such coating materials are in general, provides good sealing properties and excellent barrier against humidity. Additionally, the coating method is based on a melt coating process where no aqueous solution is involved during the process; thus, a microencapsulated product with very low water content and water activity can be obtained at the end of the process in addition to the very short coating time duration which is of the interest. However, this technology suffers from some significant shortcomings. These include: I. poor mechanical properties of such coating layers where mono- and di-glycerides of saturated fatty acids, polyglycerols esterified with saturated fatty acids, free saturated fatty acids and glyceryl dipalmitostearate are used alone, specially due to the development of stresses in the film leading to pores and cracks formation, mainly during the shelf life, and consequently imperfection in barrier and low sealing properties. II. Poor adhesion onto the highly hydrophilic surface, especially due to the high interfacial tension, and consequently incomplete coating during the coating process, which is an additional deficiency of these coating materials where they are purely used as the coating material for microencapsulation. III. Incomplete dissolution and the release of active material from the core after administration/consumption in the digestion system, specially where a thick coating is applied.

Penhasi Adel and Alon Shiran disclose (U.S. Ser. No. 10/543,175 B1) A film composition for coating a pharmaceutical, nutraceutical or nutritional composition comprising a molecular mixture of a hydrophilic film forming polymer having thermo-sensitive sol gel forming properties and having a first lower critical solution temperature (LCST), a hydrophobic fatty component and a micelle-forming block copolymer having a second lower critical solution temperature (LCST) and an HLB value of about 9 to 20, wherein said second LCST is lower than said first LCST. This film composition demonstrated a very good barrier against high humidity and moisture and thus excellent protection at room temperature using a single coating layer applied on a dosage form (Adel Penhasi, Moran Elias, Efrat Eshtauber, Hadar Naiman-Nissenboim, Albert Reuveni, Israel Baluashvili, “A novel hybrid solid dispersion film coat as a moisture barrier for pharmaceutical applications”, Journal of Drug Delivery Science and Technology, Volume 40, 2017, 105-115). Additionally, this coating system has improved flexibility and mechanical properties, and presents very good dissolution properties for complete release of the active material after oral administration. However, the coating process is based on an aqueous coating solution which leads to free water's entrapment between the coating layers and in the hydrophobic fatty component's crystals which eventually may harm the included bacteria in the dosage form during the Parker test (an accelerated test, especially designed for those dosage forms containing probiotic bacteria, which is carried out at 37° C. for four weeks). Therefore, this technology is not appropriate for highly sensitive bacteria to water activity such as lactobacillus Rhamnosus. Likewise, the coating process is still very long which results in the waste of energy and consequently relatively high production cost.

Penhasi Adel and Baluashvili Israel disclose [WO2019202604] a moisture resistant probiotic microcapsule comprising a core comprising probiotic microorganisms; and a coating layer comprising a hybrid solid dispersion comprising an edible fatty acid evenly dispersed within a water-soluble film forming polymer and an edible mediator. This film composition demonstrated a very good barrier against high humidity and moisture and thus excellent protection at room temperature. Additionally, this coating system has improved flexibility and mechanical properties, and presents very good dissolution properties for complete release of the active material after oral administration.

However, the coating process is based on an aqueous coating solution which leads to free water's entrapment between the coating layers and in the hydrophobic fatty component's crystals which eventually may harm the included bacteria in the dosage form during the Parker test (an accelerated test, especially designed for those dosage forms containing probiotic bacteria, which is carried out at 37° C. for four weeks). Therefore, this technology is not appropriate for highly sensitive bacteria to water activity such as Lactobacillus rhamnosus. Likewise, the coating process is still very long which results in the waste of energy and consequently relatively high production cost.

### SUMMARY OF THE INVENTION

According to some embodiments, there is provided herein a probiotic granule comprising: a core comprising probiotic bacteria; and a continuous solid dispersion coating layer coating said core comprising at least one an edible hydrophobic solid component such as fat, wax, phospholipid or a fatty acid having a melting point higher than 30° C.; at least one edible water-soluble polymeric stress absorber, dispersed within said hydrophobic solid component.

According to some embodiments, the at least one edible water-soluble polymeric stress absorber is in the form of particles having a size of between 5-200 μm, preferably, a mean average size of 150 μm or less.

According to some embodiments, the at least one edible water-soluble polymeric stress absorber has an impact force of 100-1000 N/m.

According to some embodiments, the at least one edible water-soluble polymeric stress absorber may be selected from the group including Starches, Gums, Cellulose ethers and Vinyl polymers.

According to some embodiments, the hydrophobic solid component may have a melting point higher than 30° C. and below 80° C.

According to some embodiments, the hydrophobic solid component may be stearic acid. According to some embodiments, the solid dispersion coating layer is in an amount of 10-50% weight gain (WG).

According to some embodiments, the weight percentage of the edible water-soluble polymeric stress absorber in the solid dispersion coating layer may be between 1% and 20% w/w.

According to some embodiments, the granule of the present invention may further include a second layer, which may include HPC or an enteric polymer.

According to some embodiments, the enteric polymer may include an edible polymer selected from the group including pH-sensitive polymers, for example, acid phthalate of carbohydrates, amylose acetate phthalate, cellulose acetate phthalate (CAP), other cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS), methylcellulose phthalate (MCP), polyvinyl acetate phthalate (PVAcP), polyvinyl acetate hydrogen phthalate, sodium CAP, starch acid phthalate, cellulose acetate trimellitate (CAT), styrene and maleic acid copolymers, styrene-maleic acid dibutyl phthalate copolymer, styrene-maleic acid/polyvinylacetate phthalate copolymer, polyacrylic acid derivatives such as acrylic acid and acrylic ester copolymers, polymethacrylic acid and esters thereof, polyacrylic and methacrylic acid copolymers, polyacrylic acid derivatives such as particularly copolymers comprising acrylic acid and at least one acrylic acid ester, Eudragit STM (poly(methacrylic acid, methyl methacrylate)1:2); Eudragit LTM which is an anionic polymer synthesized from methacrylic acid and methacrylic acid methyl ester), Eudragit L100TM (poly(methacrylic acid, methyl methacrylate)1:1); Eudragit L30DTM, (poly(methacrylic acid, ethyl acrylate)1:1); and Eudragit L100-55TM (poly(methacrylic acid, ethyl acrylate)1:1), polymethyl methacrylate blended with acrylic acid and acrylic ester copolymers, alginic acid and alginates such as ammonia alginate, sodium, potassium, magnesium or calcium alginate or a combination thereof.

According to some embodiments, there is provided herein a process for the production of a probiotic granule, wherein the process includes: forming a core using a hot melt process including: I. Mixing probiotic bacteria with sugar particles II. Melting at least one edible solid fat, wax or fatty acid; III. Spraying the melt of said at least one edible solid fat, wax or fatty acid onto the mixture of said probiotic bacteria with sugar particles to form solid probiotic particles (the core); coating the solid probiotic particles (the core) including: I. Melting at least one an edible hydrophobic solid component having a melting point higher than 30° C. to form a hydrophobic film; II. Adding and mixing at least one edible water-soluble polymeric stress absorber with the melt of said at least one an edible hydrophobic solid component to yield a melt of hydrophobic mixture; III. Spraying the melt of the hydrophobic mixture onto the core to provide a probiotic granule.

According to some embodiments, the process may further include coating the probiotic granule with a second layer, an enteric coating comprising an edible enteric polymer to further provide protection through GI tract.

According to some embodiments, there is provided herein a use of the granule for the preparation of a food product

According to some embodiments, there is provided herein a food product comprising the granule of the present invention, selected from the group including grains and grain products, dairy products, and sweets.

According to some embodiments, the sweets may be selected from the group including candies, jelly candies, gummies, lollipops, chewing gums, caramels, chocolate candies and chocolate.

### BRIEF DESCRIPTION OF THE DRAWINGS

FIG. 1 is a schematic illustration of a process for the production of the probiotic granule of the present invention, in accordance with some demonstrative embodiments.

FIG. 2 is an illustration of a cross section view of the probiotic granule of the present invention, in accordance with some demonstrative embodiments.

FIGS. 3-8 depict graphs of particles size distribution of probiotic particles according to some demonstrative embodiments of the present invention.

### DETAILED DESCRIPTION OF THE INVENTION

According to some demonstrative embodiments, there is provided herein a probiotic granule comprising: a core comprising probiotic bacteria; a single continuous coating layer coating said core comprising at least one an edible hydrophobic solid component such as fat, wax, phospholipid or a fatty acid having a melting point higher than 30° C.; at least one edible water-soluble polymeric stress absorber, dispersed within said hydrophobic solid component.

According to some embodiments, the at least one an edible hydrophobic solid may be in the form of a solid dispersion. According to some embodiments, the term “solid dispersion” as used herein may refer to a dispersion of at least one edible water-soluble polymeric stress absorber in a solid matrix of the at least one an edible hydrophobic solid.

According to some embodiments, the unique structure of the hydrophobic solid dispersion provides some surprising effects, including, for example:

Protection to the bacteria during the encapsulation process—according to some embodiments, the encapsulation process may be harmful to the bacteria on its own. For example, a core bearing the bacteria may be exposed to heat and/or humidity when the coating is sprayed on the core. The unique hydrophobic characteristics of the coating enable the protection of the core by repelling humidity during the delicate stage of coating.

Protection from heat and/or humidity during storage—According to some embodiments, and as exemplified below, the unique structure of the granule of the present invention, comprising both a probiotic core and a solid dispersion coating which includes at least one edible water-soluble polymeric stress absorber dispersed within a hydrophobic solid component, enables the survival of the probiotics for prolonged periods of time and storage, for example, 1-60 months, depending on packaging materials and storage conditions.

Protection from the extremely acidic conditions of the digestive system—According to some embodiments, the probiotics are to be ingested, and as such need to survive the conditions present in the GI tract, specifically, the acidic environment of the stomach.

According to these embodiments, the unique coating of the granule of the present invention may provide protection against the harmful conditions of the stomach.

According to some embodiments, an additional (second) layer of protection may be layered on top of the solid dispersion layer.

According to some embodiments, the second layer may provide an additional protection to the granule, e.g., on top of the protection provided by the solid dispersion layer. For example, according to some embodiments the second layer may be a protective layer against humidity or heat including for example, HPC, or according to other embodiments, the second layer may provide protection against harmful conditions in the GI tract, for example, including an enteric coating, as explained in detail below.

Controlled release properties—According to some demonstrative embodiments, the hydrophobic character of the coating of the granule of the present invention, prevents the immediate release of the bacteria from the granule. However, it is important that the stress absorber would be a water soluble molecule as the existence of a water soluble stress absorber dispersed within the hydrophobic solid component may create pores within the hydrophobic solid component, enabling the penetration of water into the coating thereby enhancing the dissolution.

According to some embodiments, the concentration/amount of the stress absorber may affect the rate of dissolution of the coating, for example, the higher the concentration or ratio of the stress absorber in the solid dispersion the higher the dissolution rate of the solid dispersion.

According to some demonstrative embodiments, the solid dispersion coating the core may be in an amount of 10-50% weight gain (WG). According to these embodiments, the amount or concentration of the solid dispersion is preferably between 10-50% weight gain (WG) of the granule weight, as this enables an optimal balance between the minimum protection required to the granule and the effective dissolution of the solid dispersion layer, thereby enabling the release of the probiotics upon consumption.

According to some demonstrative embodiments, the core may comprise the probiotic bacteria in the form of one or more probiotic particles, wherein the particles may be combined together and may be at least partially coated by at least one edible solid fat, wax, phospholipid or fatty acid.

According to some demonstrative embodiments, the probiotic particles may be at a size of between 10-1000 μm, preferably between 100-850 μm.

According to some demonstrative embodiments of the present invention, the unique granule of the present invention allows for the coating of the probiotic particles and/or the core while obviating the need to use water in the process. According to some embodiments, the existence of water during the manufacturing or coating process may cause harm to the probiotics, which are sensitive to environmental conditions such as humidity and water activity.

According to some embodiments, the surprising effect of the manufacturing process of the present invention as described hereinbelow, allows for a stable probiotic granule having viable probiotic bacteria.

According to some embodiments, viability is an inherent property of probiotics since the current definition of probiotics, issued by the Joint FAO/WHO Working Group, defines that probiotics are ‘live microorganisms which, when administered in adequate amounts, confer a health benefit on the host’.

According to some demonstrative embodiments, there is provided herein a unique coating of a probiotic core, wherein the coating includes a solid dispersion of a at least one edible water-soluble polymeric stress absorber dispersed within at least one an edible hydrophobic solid.

According to some demonstrative embodiments, the stress absorber allows for the mechanical protection of the granule (and the probiotics contained within) as well as impacting on the dissolution of the granule, as explained herein. According to some demonstrative embodiments, the stress absorber of the present invention may include a unique set of characteristics including being water soluble, edible and having a high impact force of 100-1000 N/m, preferably 200-800 and most preferably 250-700 N/m.

According to some embodiments, the impact force, also known as impact resistance (toughness) of a polymer depends on both intrinsic and extrinsic factors, for example, some intrinsic factors may include molecular structure, molecular weight (distribution), cohesive energy and morphology (crystallinity and crystal structure), whereas some extrinsic factors may include temperature applied stress and the like.

According to some embodiments, the at least one edible water-soluble polymeric stress absorber is in the form of particles having a size of between 5-200 μm, preferably, a mean average size of 150 μm or less. According to some embodiments of the present invention, the desired size of the stress absorber, as described herein, allows for the effective application of the absorber, e.g, when sprayed and when passing through a nozzle, and at the same time for achieving the desired purpose of enhancing and controlling the disintegration of the solid dispersion layer.

According to some embodiments, the at least one edible water-soluble polymeric stress absorber is selected from the group including:

Starches such as native starch, pregelatinized starch, partially gelatinized starch, hydrolyzed starch such as dextrin (E1400), acid-treated starch (E1401), alkaline-treated starch (E1402), bleached starch (E1403), oxidized starch (E1404), enzyme-treated starch (E1405), monostarch phosphate (E1410), distarch phosphate (E1412), phosphated distarch phosphate (E1413), acetylated distarch phosphate (E1414), starch acetate (E1420), acetylated distarch adipate (E1422), hydroxypropyl starch (E1440), hydroxypropyl distarch phosphate (E1442), hydroxypropyl distarch glycerol (E1443), starch sodium octenyl succinate (E1450), acetylated oxidized starch (E1451);

Gums such as natural gums obtained from botanical resources such as arabic gum (E414), tragacanth gum (E413), karaya gum (E416), ghatti gum, guar gum (E412) from guar beans, carob (locust) bean gum (E410), konjac gum (E425), tara gum (E417), pectin and its derivatives, natural gums obtained from seaweeds such as agar (E406), alginic acid (E400) and sodium alginate (E401), carrageenan (E407), etc., natural gums produced by bacterial fermentation such as gellan gum (E418) and xanthan gum (E415);

Cellulose ether (cellulose derivatives)—The commercially important properties of cellulose ethers are determined by their molecular weights, chemical structure and distribution of the substituent groups, degree of substitution and molar substitution (where applicable). These properties generally include solubility, viscosity in solution, surface activity, thermoplastic film characteristics and stability against biodegradation, heat, hydrolysis and oxidation. Viscosity of cellulose ether solutions is directly related with their molecular weights. Examples of mostly used cellulose ethers are: Methyl cellulose (MC), Ethyl cellulose (EC), Hydroxyethyl cellulose (HEC), Hydroxypropyl cellulose (HPC), hydroxypropylmethyl cellulose (HPMC), carboxymethyl cellulose (CMC) and sodium carboxymethyl cellulose (NaCMC);

Vinyl polymers such as polyvinyl alcohol; and/or a combination of all of the above. According to some embodiments, the weight percentage of the edible water-soluble polymeric stress absorber in the solid dispersion coating layer (also referred to as “first fatty coating layer”) ranges between 1% and 20%, preferably between 2% and 15% and most preferably between 5% and 10% w/w.

According to these embodiments, the weight percentage of the edible water-soluble polymeric stress absorber in the solid dispersion coating layer described herein allows for the effective disintegration of the solid dispersion layer due to the hydrophilic areas in the solid dispersion coating layer (which is hydrophobic), and also for the effective controlled release properties of this layer, and yet still allow for the effective protection of the core throughout the preparation process of the granule and the later stages of storage and transport.

According to some embodiments, the successful balance between the protection of the probiotics during production and storage and the ability to effectively release the probiotics upon consumption is a result of the unique structure of the granule described in the present invention.

According to some embodiments, the at least one edible water-soluble polymeric stress absorber may preferably be a starch and more preferably a starch with a rich source of amylose, for example, including amylose in a concentration of 25-85% w/w, preferably at least 35% w/w of Amylose. According to these embodiments, for example, a suitable Amylose rich starch may be pea starch.

According to some embodiments, the coating layer may further comprise a mediator selected from the group including a glidant, anticaking and/or lubricant such as sodium carbonate (E500), tricalcium phosphate (E341), potassium carbonate (E501), ammonium carbonate (E503), magnesium carbonate (E504), hydrochloric acid (E507), potassium chloride (E508), calcium chloride (E509), ammonium chloride (E510), magnesium chloride (E511), stannous chloride (E512), sulphuric acid (E513), sodium sulphates (E514), potassium sulphate (E515), calcium sulphate (E516), ammonium sulphate (E517), magnesium sulphate, epsom salts (E518), copper sulphate (E519), aluminium sulphate (E520), aluminium sodium sulphate (E521), aluminium potassium sulphate (E522), aluminium ammonium sulphate (E523), sodium hydroxide (E524), potassium hydroxide (E525), calcium hydroxide (E526), ammonium hydroxide (E527), magnesium hydroxide (E528), calcium oxide (E529), magnesium oxide (E530), sodium ferrocyanide (E535), potassium ferrocyanide (E536), calcium ferrocyanide (E538), dicalcium diphosphate (E540), sodium aluminium phosphate (E541), bone phosphate (E542), edible bone phosphate (derived from steaming animal bones and used as anti-caking agent, emulsifier and source of phosphorous in food supplements.), sodium silicate (E550), silicon dioxide (E551) (Fumed silica), calcium silicate (E552), (i) magnesium silicate and (ii) magnesium trisilicate (E553(a)), talc (E553(b)), sodium aluminum silicate (E554), potassium aluminum silicate (produced from several natural minerals.) (E555), aluminum calcium silicate (produced from several natural minerals.) (E556), bentonite (a natural type of clay from volcanic origin. it is a decolorising agent, filter medium, emulsifier and anti-caking agent. bentonite is used in pharmaceutical agents for external use, edible fats and oils, sugar, wine.) (E558), kaolin (E559), magnesium stearate, calcium stearate (E572), gluconic acid (E574), glucono delta-lactone (E575), sodium gluconate (E576), potassium gluconate (E577), calcium gluconate (E578), ferrous gluconate (E579), ferrous lactate (E585), polydimethylsiloxane (E900) and a combination thereof.

According to some embodiments, the mediator may preferably silicon dioxide (E551) (Fumed silica).

According to some embodiments, the mediator may be in the form of particles having a size ranging between 20 to 150 μm, for example, having a mean average size of 100 μm.

According to some demonstrative embodiments, the hydrophobic solid component may have a melting point higher than 30° C., preferably, below 80° C. According to these embodiments, the specific melting point of the hydrophobic component is designed to enable the granules of the present invention to be stable at room temperature. According to some embodiments, when the temperature exceeds 80° C. the tension within the formulation increases and requires additional amounts of stress absorber.

According to some demonstrative embodiments, the solid dispersion coating the core may preferably include an amount sufficient to cause 10-50% weight gain to the granule upon coating of the granule.

According to some embodiments, the granule may further comprise a second layer, wherein said second layer may include at least one enteric layer comprising an edible polymer.

According to some embodiments, the enteric layer may provide additional protection to the granule of the invention through GI tract destructive parameters such as low pHs and enzymes.

According to some embodiments, the edible polymer of the enteric layer may be selected from the group including, for example, acid phthalate of carbohydrates, amylose acetate phthalate, cellulose acetate phthalate (CAP), other cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS), methylcellulose phthalate (MCP), polyvinyl acetate phthalate (PVAcP), polyvinyl acetate hydrogen phthalate, sodium CAP, starch acid phthalate, cellulose acetate trimellitate (CAT), styrene and maleic acid copolymers, styrene-maleic acid dibutyl phthalate copolymer, styrene-maleic acid/polyvinylacetate phthalate copolymer, polyacrylic acid derivatives such as acrylic acid and acrylic ester copolymers, polymethacrylic acid and esters thereof, polyacrylic and methacrylic acid copolymers, polyacrylic acid derivatives such as particularly copolymers comprising acrylic acid and at least one acrylic acid ester, Eudragit STM (poly(methacrylic acid, methyl methacrylate)1:2); Eudragit LTM which is an anionic polymer synthesized from methacrylic acid and methacrylic acid methyl ester), Eudragit L100TM (poly(methacrylic acid, methyl methacrylate)1:1); Eudragit L30DTM, (poly(methacrylic acid, ethyl acrylate)1:1); and Eudragit L100-55TM (poly(methacrylic acid, ethyl acrylate)1:1), polymethyl methacrylate blended with acrylic acid and acrylic ester copolymers, alginic acid and alginates such as ammonia alginate, sodium, potassium, magnesium or calcium alginate or a combination thereof.

According to some demonstrative embodiments, the term “continuous” refers to an unbroken, wholesome coverage of the core by the coating layer, for example, to ensure the prevention and/or diminishment of harmful conditions to the core such as any conditions that may harm the bacteria, e.g., heat and humidity.

According to some embodiments, the probiotic bacteria may be any suitable live microorganisms which when administered in adequate amounts confer a health benefit on the host.

Examples of probiotic bacteria may include but are not limited to Bacillus coagulans GBI-30, 6086, Bacillus subtilis var natt, Bifidobacterium LAFTI® B94, Bifidobacterium sp LAFTI B94, Bifidobacterium bifidum, Bifidobacterium bifidum rosell-71, Bifidobacterium breve, Bifidobacterium breve M-16V, Bifidobacterium breve Rosen-70, Bifidobacterium infantis, Bifidobacterium lactis, Bifidobacterium longum, Bifidobacterium longum Rosen-175, Bifidobacterium animalis, Bifidobacterium animalis spp. lactis NCC 2818 (BL818), Bifidobacterium animalis subsp. lactis BB-12, Bifidobacterium animalis subsp. lactis HN019, Bifidobacterium animalis spp. lactis NH019 (HOWARLP Bifido 300B), Bifidobacterium infantis 35624, Escherichia coli M-17, Escherichia coli Nissle 1917, Lactobacillus acidophilus, Lactobacillus acidophilus LAFTI® L10, Lactobacillus acidophilus LAFTI L10, Lactobacillus casei LAFTI® L26, Lactobacillus casei LAFTI L26, Lactobacillus brevis, Lactobacillus bulgaricus, Lactobacillus casei, Lactobacillus gasseri, Lactobacillus paracasei, Lactobacillus plantarum, Lactobacillus reuteri ATTC 55730 (Lactobacillus reuteri SD2112), Lactobacillus rhamnosus, Lactobacillus rhamnosus GG (ATCC 53103), Lactobacillus salivarius, Lactobacillus delbrueckii, Lactobacillus fermentum, Lactococcus lactis, Lactococcus lactis subsp, Lactococcus lactis Rosen-1058, Lactobacillus paracasei St11 (or NCC2461] Lactobacillus fortis Nestle, Lactobacillus johnsonii Lal (=Lactobacillus LC1, Lactobacillus johnsonii NCC533) Nestle, Lactobacillus johnsonii 456, Lactobacillus rhamnosus Rose11-11, Lactobacillus acidophilus Rosen-52, Lactobacillus paracasei Streptococcus thermophilus, Diacetylactis, Saccharomyces cerevisiae, and a mixture thereof.

According to a preferred embodiment of the invention, the probiotic bacteria in the core of the granules may be mixed with at least one sugar and/or at least one oligosaccharide or polysaccharides (as a supplemental agent for the bacteria), and optionally other food grade additives such as stabilizers, fillers, binders, surfactant etc.

The sugar may comprise monosaccharides such as trioses including ketotriose (dihydroxyacetone) and aldotriose (glyceraldehyde), tetroses such as ketotetrose (erythrulose), aldotetroses (erythrose, threose) and ketopentose (ribulose, xylulose), pentoses such as aldopentose (ribose, arabinose, xylose, lyxose), deoxy sugar (deoxyribose) and ketohexose (psicose, fructose, sorbose, tagatose), hexoses such as aldohexose (allose, altrose, glucose, mannose, gulose, idose, galactose, talose), deoxy sugar (fucose, fuculose, rhamnose) and heptose such as (sedoheptulose), and octose and nonose (neuraminic acid), multiple saccharides such as 1) disaccharides, such as sucrose, lactose, maltose, trehalose, turanose, and cellobiose, 2) trisaccharides such as raffinose, melezitose and maltotriose, 3) tetrasaccharides such as acarbose and stachyose, 4) other oligosaccharides such as fructooligosaccharide (FOS), galactooligosaccharides (GOS) and mannan-oligosaccharides (MOS), 5) polysaccharides such as glucose-based polysaccharides/glucan including glycogen starch (amylose, amylopectin), cellulose, dextrin, dextran, beta-glucan (zymosan, lentinan, sizofiran), and maltodextrin, fructose-based polysaccharides/fructan including inulin, levan beta 2-6, mannose-based polysaccharides (mannan), galactose-based polysaccharides (galactan), and N-acetylglucosamine-based polysaccharides including chitin. Other polysaccharides may be comprised, including gums such as arabic gum (gum acacia).

According to some embodiments, the granule of the present invention may be admixed with and/or incorporated into food, feed, food supplement and infant food. According to some embodiments the food products (foodstuffs) may include but not limited to creams, biscuits creams, biscuit fill-in, chocolates, sauces, mayonnaise, dairy products such as milk and yogurt, etc.

According to some embodiments, the granule of the present invention may be consumed and still provide probiotics with high survival/viability and resistance during processing (e.g. heat tolerance) and post production during storage of the foodstuff.

According to some embodiments, the probiotic microorganisms such as probiotic bacteria (probiotics) may have improved stability at ambient temperature and thus an extended shelf life.

According to some embodiments, the granule may be provided for consumption in different dosage forms such as hard gelatin capsule, sachet and tablets.

According to some demonstrative embodiments, the granule of the present invention (also referred to herein as “the composition”) provides probiotic bacteria that are stable for long periods of time at ambient temperatures, and varying degrees of humidity and water activity values.

The present invention in fact overcomes the shortcomings of the prior arts by presenting distinctive properties over suggested/invented technologies; for example, I. The coating layer of microcapsule according to the present invention, has improved mechanical properties due to presence of the polymeric filler acting as stress-absorbent that absorbs the stress evolved through the thickness of the coating layer and where the formation of pores and cracks over the coating during the shelf life is totally eliminated, II. The coating process is based on a melt coating process which dismisses the use of aqueous-based solutions which in turn in one hand leads to a very short time's duration of the process and consequently an affordable production cost, and on the other hand, results in a product with very low values of water activity and consequently improved shelf life and enhanced stability during a Parker test, III. Improved adhesion onto highly hydrophilic cores due to the presence of the polymer in the coating, IV. Good dissolution properties for complete release of bacteria after consumption due to the presence of the water-soluble polymer in the coating layer, V. surprisingly a reduced particle size and particle size distribution achieved after the microencapsulation using the formulation according to the present invention.

According to some embodiments, the water activity in the resulting granule of the present invention is between 0.0001 and 0.3 a_{w}, preferably between 0-0.2 a_{w}.

According to some embodiments, the granule of the present invention may be in a size ranging from 15 μm to 2000 μm, preferably between 50 μm to 1000 μm.

According to some embodiments, the unique structure of the granule of the present invention includes a core with probiotics, surrounded by a continuous single coating layer, wherein this structure creates a granule with superior properties, when compared, for example, to granules having multiple coating layers. For example, such superior properties may include a uniform PSD of the granules, wherein a uniform PSD allows for optimal mixing of the granules into other powders, e.g., powder infant formulas, protein powders and the like.

Optimal mixing may refer to minimal segregation when the granules are mixed with another power, resulting in better rheology and flow of the powder.

In addition, as per some embodiments of the invention, the granule of the present invention, possessing a core and a single continuous coating layer has superior dissolution properties, when compared to a granule having two or more separate coating layers. According to some embodiments, “superior dissolution properties” may refer to one or more of the following dissolution indicators such as rate of dissolution, diminishment of peaks and valleys in the extent of dissolution, i.e., a constant and/or expected dissolution, coherence of dissolution of the granules of the invention with the dissolution of any additional powder added to the granules, etc.

According to some embodiments, the granule of the present invention comprises probiotic cultures that are substantially stable at room temperature and to high humidity conditions with no need for refrigeration or storage in vacuum or in an oxygen free environment.

According to some embodiments, the term “infant formula” may refer to any suitable infant food product such as powdered infant formula and ready to use liquid infant formula.

According to some embodiments, there is provided herein a process for preparing a nutritionally acceptable composition comprising probiotic microorganisms, the composition being resistant to reconstitution temperatures (which is up to 75° C.) as recommended by WHO/FAO (70° C.) for safe preparation of powdered infant formula, oxygen, humidity, and a relatively high water activity said composition being added to an infant food product such as powdered infant formula.

According to some embodiments, the granule of the present invention may be added a food product undergoing heating up to 75° C. (such as a pasteurization process) during its preparation, wherein the bacteria encompassed within said granule remains viable despite the pasteurization process and despite being exposed to high temperatures for a certain period of time, for example, up to 1 minute.

According to some embodiments, there is provided herein a probiotic granule capable of withstanding heat, oxygen, humidity and water activity comprising: (a) a core composition in form of solid particles or granulate containing probiotic bacteria and at least one sugar compound such as maltodextrin, trehalose, lactose, galactose, sucrose, fructose and alike, and at least one edible solid fat, wax, phospholipid or fatty acid, forming a stable hydrophobic film or matrix which embeds the probiotic or forms film around the probiotics, optionally a stabilizer such as oxygen scavenger containing L-cysteine base or hydrochloride, and optionally other food grade ingredients such as a filler a surfactant and binder where the total amount of probiotics in the mixture is from about 10% to about 90% by weight of the core composition (b) a coating layer which coats the said core particles, comprising I. at least one an edible hydrophobic solid component such as fat, wax, phospholipid or fatty acid having a melting point higher than 30° C. forming a stable hydrophobic film around the probiotics core particles, II. At least one edible water soluble polymeric stress absorber, dispersed within said hydrophobic solid component to provide the said hydrophobic solid component with mechanical stability and enhanced dissolution after digestion for complete release of included probiotic bacteria, III. Optionally, an edible mediator, wherein said edible mediator is a glidant, anticaking and or lubricant. (c) Optionally an edible enteric polymer which may further provide protection through GI tract destructive parameters such as low pHs and enzymes.

According to some embodiments, the granules (also referred to as “microparticles”, “microparticulates” or microcapsules, or microspheres) may be used for admixing/adding to food products such as chocolate, cheese, creams, sauces, mayonnaise, dairy products, biscuit fill-in, dietary supplement product, infant food, and feed, said probiotic particles are capable of resisting against oxygen, humidity, water activity, and temperature to provide stabilized probiotic bacteria.

According to some embodiments, the stabilized bacteria are capable of resisting high temperature, humidity, water activity and oxygen, e.g., during manufacturing or preparation process where there is exposure to such harmful conditions. The stabilized bacteria are further capable of resisting harmful conditions during storage at ambient temperature even after they are added to a food product under humid environment and oxygen. An example of high temperature to be resisted is a tempering step during preparation of chocolate, or mixing within cream compositions or pasteurization of dairy products, or pasteurization of liquid infant formula, or reconstitution of powdered infant formula according to the new WHO/FAO guidelines (WHO, 2007 Safe preparation, storage and handling of powdered infant formula. www.who.int/foodsafety/publications/micro/pif_guidelines.pdf (reconstitution at 70° C.).

The invention relates to a process for the preparation of a food, feed, and infant food product comprising a heating step, the product containing active probiotic bacteria, comprising i) preparing stabilized probiotic granules according to claim 1; ii) admixing said stabilized probiotic granules into a semi-final product; iii) heating the mixture of said probiotic granules and semi-final product at a predetermined temperature and for a predetermined time period; and vi) completion of said liquid based semi-final product containing said stabilized probiotic granules by cooling down said mixture, thereby obtaining said final product containing stabilized active probiotic bacteria showing high stability during the storage and shelf life of the final product. The term “semi-final product” describes a stage in the preparation of a food product according to the invention, in which said food product does not yet contain all the components or has not yet passed all the preparation steps, being not yet ready for the consumption.

The invention provides stabilized probiotic particles/or microcapsules for admixing to a food product, resistant to ambient temperature, humidity and oxygen, comprising a core or granules, containing probiotic bacteria and at least one sugar compound such as maltodextrin, trehalose, lactose, galactose, sucrose, fructose and alike, a stabilizer such as oxygen scavenger containing L-cysteine base or hydrochloride and alike, a filler such as microcrystalline cellulose, and optionally other food grade ingredients such as a surfactant and binder, which can be prepared by a hot melt granulation where a melt of a solid fat or wax, phospholipid or fatty acid or a combination thereof is used as binder. If the initial particle size of probiotics and other excipients included in the core is sufficient to enable the coating of the first coating layer, no granulation process is needed.

According to some embodiments, the food product according to the invention may be a product having the form of liquid, suspension, emulsion, paste, powder, flax and granules. In a preferred embodiment, said particles comprise (a) a core or granule composition in form of solid particles or granulate containing probiotic bacteria and at least one sugar compound such as maltodextrin, trehalose, lactose, galactose, sucrose, fructose and alike, and at least one edible solid fat, wax, phospholipid or fatty acid, forming a stable hydrophobic film or matrix which embeds the probiotic or forms film around the probiotics, optionally a stabilizer such as oxygen scavenger containing L-cysteine base or hydrochloride, and optionally other food grade ingredients such as a filler a surfactant like tween 80 and binder where the total amount of probiotics in the mixture is from about 10% to about 90% by weight of the core composition.

(b) a first hydrophobic coating layer which coats the said core particles, comprising I. at least one an edible hydrophobic solid component such as fat, wax, phospholipid or fatty acid having a melting point higher than 30° C. forming a stable hydrophobic film around the probiotics core particles, II. At least one edible water-soluble polymeric stress absorber, dispersed within said hydrophobic solid component to provide the said hydrophobic solid component with mechanical stability and an enhanced dissolution after digestion for complete release of included probiotic bacteria, III. Optionally, an edible mediator, wherein said edible mediator is a glidant, anticaking and or lubricant.

(c) Optionally an edible enteric polymer which may further provide protection through GI tract destructive parameters such as low pHs and enzymes.

The invention provides a food product selected from such as creams, biscuits creams, biscuit fill-in, chocolates, sauces, cheese, mayonnaise, dairy products such as milk and yogurt, infant food such as powdered infant formula and ready-to-use infant formula (liquid infant formula), dietary supplement products and etc, which product is a health food product comprising probiotic bacteria which are stabilized as described above for long term storage and shelf life. The invention, thus, relates to healthy food beneficially affecting the consumer's intestinal microbial balance, wherein said heat-resistance and heat-processability are ensured by coating probiotic cores by layers which limit the transmission of heat, oxygen and humidity to the probiotic bacteria and so increase their resistance during preparation process and storage and thus extend the shelf life.

It has now been found that probiotic bacteria may be surprisingly efficiently stabilized during shelf life or using in a food preparation process by a unique combination of some ingredients in the core and coating layer of a microcapsule structure including the probiotic bacteria, creating stabilized probiotic particles. The bacteria were formulated in a core or granule coated with at least a coating layer, thereby obtaining probiotic compositions providing viable probiotic organisms even after a prolonged time of storage at ambient temperature at high humidity, the composition being further stable on storage and shelf life of the food stuff containing the protected probiotics according to the present invention and capable of administering viable bacteria to the gastrointestinal tracts after the oral administration. The invention provides granular/particular/microcapsules probiotics to be used as healthy food additives. The present invention is particularly directed to a process for the preparation of protected probiotics against oxygen and humidity (water vapor) and water activity for incorporating into foodstuffs such as creams, biscuits creams, biscuit fill-in, chocolates, sauces, cheese, mayonnaise, infant food, feed, and dietary supplement product, etc.

The present invention provides a process for the preparation of heat, oxygen water activity and humidity resisting probiotic bacteria for a food product, infant food product or dietary supplement product, comprising:

(a) a core or granule composition in form of solid particles or granulate containing probiotic bacteria and at least one sugar compound such as maltodextrin, trehalose, lactose, galactose, sucrose, fructose and alike, and at least one edible solid fat, wax, phospholipid or fatty acid, forming a stable hydrophobic film or matrix which embeds the probiotic or forms film around the probiotics, optionally a stabilizer such as oxygen scavenger containing L-cysteine base or hydrochloride, and optionally other food grade ingredients such as a filler a surfactant like tween 80 and binder where the total amount of probiotics in the mixture is from about 10% to about 90% by weight of the core composition.

(b) a first hydrophobic coating layer which coats the said core particles, comprising I. at least one an edible hydrophobic solid component such as fat, wax, phospholipid or fatty acid having a melting point higher than 30° C. forming a stable hydrophobic film around the probiotics core particles, II. At least one edible water-soluble polymeric stress absorber, dispersed within said hydrophobic solid component to provide the said hydrophobic solid component with mechanical stability and an enhanced dissolution after digestion for complete release of included probiotic bacteria, III. Optionally, an edible mediator, wherein said edible mediator is a glidant, anticaking and or lubricant.

(c) Optionally an edible enteric polymer which may further provide protection through GI tract destructive parameters such as low pHs and enzymes.

In an important and preferred embodiment of the invention, said stabilized probiotic particles are added to a food product such as such as creams, biscuits creams, biscuit fill-in, chocolates, sauces, cheese, mayonnaise, dairy products such as milk and yogurt, infant food such as powdered infant formula and ready-to-use infant formula (liquid infant formula), dietary supplement products, etc. In an important and preferred embodiment of the invention, said stabilized probiotic particle has I. A core or granule composition in form of solid particle or granulate containing probiotic bacteria and at least one sugar compound such as maltodextrin, trehalose, lactose, galactose, sucrose, fructose and alike, and at least one edible solid fat, wax, phospholipid or fatty acid, forming a stable hydrophobic film or matrix which embeds the probiotic or forms film around the probiotics, optionally a stabilizer such as oxygen scavenger containing L-cysteine base or hydrochloride, and optionally other food grade ingredients such as a filler a surfactant like tween 80 and binder where the total amount of probiotics in the mixture is from about 10% to about 90% by weight of the core composition prepared by a hot melt process where a melt of said edible solid fat, wax, phospholipid or fatty acid is sprayed onto a mixture of probiotic bacteria and said sugar compound, II. A first hydrophobic coating layer which coats the said core particles, comprising at least one an edible hydrophobic solid component such as fat, wax, phospholipid or fatty acid having a melting point higher than 30° C. forming a stable hydrophobic film around the probiotics core particles, and at least one edible water-soluble polymeric stress absorber, and optionally, an edible mediator, selected from the group consisting of glidant, anticaking and or lubricant, prepared by a hot melt process where a melt of said edible hydrophobic solid component into which said edible water-soluble polymeric stress absorber, and optionally, said an edible mediator are homogeneously dispersed is sprayed onto said core or granule to result in a coating layer which coats the said core particles where said water-soluble polymeric stress absorber is dispersed within said hydrophobic solid component to provide the said hydrophobic solid component with mechanical stability and an enhanced dissolution after digestion for complete release of included probiotic bacteria, and III. Optionally, an edible mediator, wherein said edible mediator is a glidant, anticaking and or lubricant. Finally, optionally an edible enteric polymer which may further provide protection through GI tract destructive parameters such as low pHs and enzymes.

In another important embodiment of the invention, said stabilized probiotic particles/or microcapsules are added to a food product such as such as creams, biscuits creams, biscuit fill-in, chocolates, sauces, cheese, mayonnaise, dairy products such as milk and yogurt, infant food such as powdered infant formula and ready-to-use infant formula (liquid infant formula), dietary supplement products, etc. where said stabilized probiotic particle has I. A core or granule composition in form of solid particle or granulate containing probiotic bacteria and at least one sugar compound such as maltodextrin, trehalose, lactose, galactose, sucrose, fructose and alike, optionally a stabilizer such as oxygen scavenger containing L-cysteine base or hydrochloride, and optionally other food grade ingredients such as a filler a surfactant like tween 80 and binder where the total amount of probiotics in the mixture is from about 10% to about 90% by weight of the core composition prepared by a hot melt process where a melt of at least one edible hydrophobic solid component such as fat, wax, phospholipid and fatty acid into which at least one edible water-soluble polymeric stress absorber, and optionally, at least one edible mediator, are homogeneously dispersed, is sprayed onto a mixture of probiotic bacteria and said sugar compound, to form a stable hydrophobic film or matrix which embeds the probiotic or forms film around the probiotics, II. A first hydrophobic coating layer which coats the said core particles, comprising at least one an edible hydrophobic solid component such as fat, wax, phospholipid or fatty acid having a melting point higher than 30° C. forming a stable hydrophobic film around the probiotics core particles, and at least one edible water-soluble polymeric stress absorber, and optionally, an edible mediator, selected from the group consisting of glidant, anticaking and or lubricant, prepared by a hot melt process where a melt of said edible hydrophobic solid component into which said edible water-soluble polymeric stress absorber, and optionally, said an edible mediator are homogeneously dispersed is sprayed onto said core or granule to result in a coating layer which coats the said core particles where said water-soluble polymeric stress absorber is dispersed within said hydrophobic solid component to provide the said hydrophobic solid component with mechanical stability and an enhanced dissolution after digestion for complete release of included probiotic bacteria, and III. Optionally, an edible mediator, wherein said edible mediator is a glidant, anticaking and or lubricant. Finally, optionally an edible enteric polymer which may further provide protection through GI tract destructive parameters such as low pHs and enzymes.

In a preferred embodiment of the invention, the stabilized probiotic particles comprising I. a core or granule containing probiotic bacteria, and at least one sugar compound such as maltodextrin, trehalose, lactose, galactose, sucrose, fructose and alike, and optionally a stabilizer such as oxygen scavenger containing L-cysteine base or hydrochloride, and optionally other food grade ingredients such as a filler a surfactant like tween 80 and binder, II. A first coating layer that coats said core or granule, containing at least one an edible hydrophobic solid component such as fat, wax, phospholipid or fatty acid having a melting point higher than 30° C., at least one edible water-soluble polymeric stress absorber, and optionally, an edible mediator such as glidant, anticaking and or lubricant, wherein said first coating layer is also used as said binder included in said core or granule which forms a stable hydrophobic film or matrix which embeds the probiotic or forms film around the probiotics. In a preferred embodiment of the invention, the stabilized probiotic particles comprising I. a core or granule containing probiotic bacteria, and at least one sugar compound such as maltodextrin, trehalose, lactose, galactose, sucrose, fructose and alike, and optionally a stabilizer such as oxygen scavenger containing L-cysteine base or hydrochloride, and optionally other food grade ingredients such as a filler a surfactant like tween 80 and binder, II. A first coating layer that coats said core or granule, containing at least one an edible hydrophobic solid component such as fat, wax, phospholipid or fatty acid having a melting point higher than 30° C., at least one edible water-soluble polymeric stress absorber, and optionally, an edible mediator such as glidant, anticaking and or lubricant, wherein said first coating layer is also used as said binder included in said core or granule which forms a stable hydrophobic film or matrix which embeds the probiotic or forms film around the probiotics, wherein the total amount of probiotics in the core is from about 10% to about 90% by weight of the core composition, total amount of said binder in the core is up to about 10% by weight of the core composition, total amount of said edible hydrophobic solid component is up to about 90% by weight of the first coating composition, total amount of said edible water-soluble polymeric stress absorber is up to about 20% by weight of the first coating composition, and total amount of said edible mediator is up to about 5% by weight of the first coating composition.

In a preferred embodiment of the invention, the stabilized probiotic particles/or microcapsules comprising I. a core or granule composition in form of solid powder or granulate containing probiotic bacteria and at least one sugar compound such as maltodextrin, trehalose, lactose, galactose, sucrose, fructose and alike, and at least one edible solid fat, wax, phospholipid or fatty acid, forming a stable hydrophobic film or matrix which embeds the probiotic or forms film around the probiotics, optionally a stabilizer such as oxygen scavenger containing L-cysteine base or hydrochloride, and optionally other food grade ingredients such as a filler a surfactant like tween 80 and binder.

II. A first coating layer that coats said core or granule, containing at least one an edible hydrophobic solid component such as fat, wax, phospholipid or fatty acid having a melting point higher than 30° C., at least one edible water-soluble polymeric stress absorber, and optionally, an edible mediator such as glidant, anticaking and or lubricant, wherein said edible solid fat, wax, phospholipid or fatty acid is used as a binder and chemically is the same as said edible hydrophobic solid component.

In a preferred embodiment of the invention, the stabilized probiotic particles/or microcapsules comprising I. a core or granule composition in form of solid powder or granulate containing probiotic bacteria and at least one sugar compound such as maltodextrin, trehalose, lactose, galactose, sucrose, fructose and alike, and at least one edible solid fat, wax, phospholipid or fatty acid, forming a stable hydrophobic film or matrix which embeds the probiotic or forms film around the probiotics, optionally a stabilizer such as oxygen scavenger containing L-cysteine base or hydrochloride, and optionally other food grade ingredients such as a filler a surfactant like tween 80 and binder. II. A first coating layer that coats said core or granule, containing at least one an edible hydrophobic solid component such as fat, wax, phospholipid or fatty acid having a melting point higher than 30° C., at least one edible water-soluble polymeric stress absorber, and optionally, an edible mediator such as glidant, anticaking and or lubricant, wherein said edible solid fat, wax, phospholipid or fatty acid is used as a binder and chemically is the same as said edible hydrophobic solid component, wherein the total amount of probiotics in the core is from about 10% to about 90% by weight of the core composition, total amount of said binder in the core is up to about 10% by weight of the core composition, total amount of said edible hydrophobic solid component is up to about 90% by weight of the first coating composition, total amount of said edible water-soluble polymeric stress absorber is up to about 20% by weight of the first coating composition, and total amount of said edible mediator is up to about 5% by weight of the first coating composition.

In a preferred process of manufacturing stabilized probiotic particles/or microcapsules, probiotic bacteria is mixed with at least one sugar compound, optionally other food grade additives such as stabilizers, fillers, binders, surfactant, etc., thereby obtaining a core mixture; particles of said core mixture are granulated using a hot melt process with a melt of at least one edible solid fat, wax, phospholipid or fatty acid thereby obtaining a core or granule composition in form of solid powder or granulate; particles of said solid powder or granulate are coated with an inner coating layer using a hot melt process with a melt of at least one edible hydrophobic solid component such as fat, wax, phospholipid or fatty acid having a melting point higher than 30° C. forming a stable hydrophobic film around the probiotics core particles in which at least one edible water-soluble polymeric stress absorber, and optionally an edible mediator, such as glidant, anticaking and or lubricant, are dispersed, thereby obtaining stabilized probiotic particles/or microcapsules wherein said hot melt process is a solvent free process and is carried out using among others a system such as Glatt hot melt fluidized bed, Romaco Innojet Ventilus, Huttlin coater/granulator, Granulex, etc, said sugar compound is selected from the group consisting of maltodextrin, trehalose, lactose, galactose, sucrose, fructose and alike and/or at least one oligosaccharide or polysaccharides such as galactan, maltodextrin, trehalose etc and/or a combination thereof, said stabilizer comprises L-cysteine base, said surfactant comprises tween 80,

(a) a core or granule composition in form of solid powder or granulate containing probiotic bacteria and at least one sugar compound such as maltodextrin, trehalose, lactose, galactose, sucrose, fructose and alike, and at least one edible solid fat, wax or fatty acid, forming a stable hydrophobic film or matrix which embeds the probiotic or forms film around the probiotics, optionally a stabilizer such as oxygen scavenger containing L-cysteine base or hydrochloride, and optionally other food grade ingredients such as a filler a surfactant like tween 80 and binder wherein the total amount of probiotics in the mixture is from about 10% to about 90% by weight of the core composition, said edible solid fat, wax or fatty acid is the said binder and said sugar compound is said filler in the core.

(b) a first fat coating layer which coats the said core particles, comprising I. at least one an edible hydrophobic solid component such as fat, wax or fatty acid having a melting point higher than 30° C. forming a stable hydrophobic film around the probiotics core particles,

II. At least one edible water-soluble polymeric stress absorber, dispersed within said hydrophobic solid component to provide the said hydrophobic solid component with mechanical stability and an enhanced dissolution after digestion for complete release of included probiotic bacteria, III. Optionally, an edible mediator, wherein said edible mediator is a glidant, anticaking and or lubricant.

(c) Optionally an edible enteric polymer which may further provide protection through GI tract destructive parameters such as low pHs and enzymes.

Stabilizer and Antioxidant (Oxygen Scavenger):

According to a preferred embodiment of the invention, the probiotic bacteria in said inner core are mixed with a stabilizer which may be selected from the group consisting of dipotassium edetate, disodium edetate, edetate calcium disodium, edetic acid, fumaric acid, malic acid, maltol, sodium edetate, trisodium edetate. According to preferred embodiments of the present invention, the core further comprises an antioxidant. Preferably, the antioxidant is selected from the group consisting of L-cysteine hydrochloride, L-cysteine base, 4,4 (2,3 dimethyl tetramethylene dipyrocatechol), tocopherol-rich extract (natural vitamin E), □-tocopherol (synthetic Vitamin E), □-tocopherol, □-tocopherol, □-tocopherol, butylhydroxinon, butyl hydroxyanisole (BHA), butyl hydroxytoluene (BHT), propyl gallate, octyl gallate, dodecyl gallate, tertiary butylhydroquinone (TBHQ), fumaric acid, malic acid, ascorbic acid (Vitamin C), sodium ascorbate, calcium ascorbate, potassium ascorbate, ascorbyl palmitate, and ascorbyl stearate. According to some embodiments of the present invention, the core further comprises both a stabilizer and an antioxidant. Without wishing to be limited by a single hypothesis or theory, stabilizing agents and antioxidants may optionally be differentiated. According to one preferred embodiment, the antioxidant is L-cysteine hydrochloride or L-cysteine base.

Filler: According to some embodiments of the present invention, the core further comprises both filler and binder. Examples of fillers include, for example, a sugar, such as lactose, glucose, galactose, fructose, or sucrose; sugar alcohols such as sorbitol, manitol, mantitol, lactitol, xylitol, isomalt, erythritol, starch including native starch, pregelatinized starch, partially gelatinized starch, chemically modified starch, enzymatically starch, hydrogenated starch hydrolysates; corn starch; and potato starch; and/or a mixture thereof. More preferably, the filler is lactose.

Surfactant: According to some embodiments of the present invention, the core further comprises a surfactant.

Edible solid fat, wax or fatty acid: According to a preferred embodiment of the invention particles of said core or granule comprise a solid fat, wax or fatty acid forming a stable hydrophobic film or matrix which embeds the probiotic. As used herein the term fats consist of a wide group of hydrophobic compounds that are generally soluble in organic solvents and largely insoluble in water. Chemically, fats are generally triesters of glycerol and fatty acids. Fats may be either solid or liquid at room temperature, depending on their structure and composition. Although the words “oils”, “fats”, and “lipids” are all used to refer to fats. Lipids are a class of macromolecules that are nonpolar and hydrophobic in nature. “Lipids” is used to refer to both liquid and solid fats, along with other related substances. Major types include fats and oils, waxes, phospholipids, and steroids. Fats, also known as triacylglycerols or triglycerides, are a kind of solid lipid. Fats are made up of fatty acids and either glycerol or sphingosine. Fatty acids may be unsaturated or saturated, depending on the presence or absence of double bonds in the hydrocarbon chain. If only single bonds are present, they are known as saturated fatty acids. Unsaturated fatty acids may have one or more double bonds in the hydrocarbon chain. Phospholipids making up the matrix of membranes, are also a class of solid lipid comprising a glycerol or sphingosine backbone to which two fatty acid chains and a phosphate-containing group are attached. Steroids are another class of lipids. Their basic structure has four fused carbon rings. Cholesterol is a type of steroid and is an important constituent of the plasma membrane, where it helps to maintain the fluid nature of the membrane. “Oil” is usually used to refer to fats that are liquids at normal room temperature, while “fats” is usually used to refer to fats that are solids at normal room temperature. An oil is defined as any of a group of natural esters of glycerol (glycerine) and various fatty acids, which are liquid at room temperature. Fats are very similar to oils and are defined as any of a group of natural esters of glycerol and various fatty acids, which are solid at room temperature and are the main constituents of animal and vegetable fat. The only discernible difference between fats and oils is their state at ambient temperature. Waxes can be ascribed as a usually solid organic compound: harder, more brittle and with a higher melting point than fats. However, some natural waxes can be soft semi-solids or even liquid.

Natural oils, fats and waxes are primarily obtained from either plant or animal sources, including sunflower, oilseed rape, oil palm, beef tallow, lanolin and beeswax. Oils and fats are organic compounds comprised of esters of glycerine and fatty acids. Glycerine is a trihydric alcohol which forms triesters with the fatty acids. The fatty acids comprise a straight carbon backbone (usually with an even number of carbon atoms) and a carboxyl group at one end.

The carbon chains can be saturated or unsaturated and vary in length from six to 22. Glycerine and fatty acids together form triglycerides.

The different types of triglyceride present give oils and fats their various properties. For example, whether the triglycerides are saturated or unsaturated affects the melting point, unsaturated triglycerides having a lower melting point than saturated triglycerides with the same carbon chain length. The triglycerides' carbon chain length also affects the melting point, with a longer chain giving a higher melting point. Waxes, being another class of lipids, are also comprised of esters. However, they mainly consist of monoesters, which are formed between a fatty alcohol molecule and a fatty acid molecule.

These monoesters range in chain length, from 16-30 carbon atoms, and are usually saturated. Natural waxes include beeswax, candelilla wax, carnauba wax, berry wax, sunflower wax, Myrica fruit wax, rice bran wax, lanolin and jojoba oil.

Examples of fats according to the present invention include fatty acids, fatty acid esters, fatty acid triesters, salts of fatty acids such as aluminum, sodium, potassium and magnesium, fatty alcohols, phospholipids, solid lipids, waxes, and a combination thereof forming a stable hydrophobic film or matrix which embeds the probiotic or forms film around the probiotics and/or a mixture of probiotic with said sugar compound particles.

According to some embodiments, at least one an edible hydrophobic solid component may be selected from the group including lauric acid, myristic acid, palmitic acid, palmitate, palmitoleate, hydroxypalmitate, arachidic acid, oleic acid, stearic acid, sodium stearat, calcium stearate, magnesium stearate, hydroxyoctacosanyl hydroxystearate, oleate esters of long-chain, esters of fatty acids, fatty alcohols, esterified fatty diols, hydroxylated fatty acid, hydrogenated fatty acid (saturated or partially saturated fatty acids), partially hydrogenated soybean, partially hydrogenated cottonseed oil, aliphatic alcohols, phospholipids, lecithin, phosphathydilcholin, triesters of fatty acids, coconut oil, hydrogenated coconut oil, cacao butter; palm oil; fatty acid eutectics; mono and diglycerides, poloxamers, block-co-polymers of polyethylene glycol and polyesters or a combination thereof.

According to some embodiments, the fatty acid of the present invention may be selected from the group including Caprylic acid, Capric acid, Lauric acid, Myristic acid, Palmitic acid, Stearic acid, Arachidic acid, Behenic acid, Lignoceric acid and Cerotic acid. The at least one an edible hydrophobic solid component is optionally and preferably at least one of stearic acid, cacao butter and a combination thereof.

According to a preferred embodiment of the invention particles of said core mixture are granulated by said solid fat, wax or fatty acid using a hot melt granulation process. In this case a melt of said solid fat, wax or fatty acid is used for granulation of said core particles therefore, said solid fat, wax or fatty acid constitutes a matrix in which said core particles are embedded thus said solid fat, wax or fatty acid functions as said binder for granulation of said core particles. According to another preferred embodiment said hot melt of said solid fat, wax or fatty acid may simultaneously constitute, by hot melt granulation process, both core binder as well as the first inner coating layer; in other words said solid fat, wax or fatty acid function as said hydrophobic solid component for coating said core as said first fat coating layer. In this case said solid fat, wax or fatty acid have a melting point higher than 30° C.

First fatty coating layer According to preferred embodiment said core particles or granule are coated by a first fatty coating layer comprising I. at least one an edible hydrophobic solid component such as fat, wax or fatty acid having a melting point higher than 30° C. forming a stable hydrophobic film around the probiotics core particles, II. At least one edible water-soluble polymeric stress absorber, dispersed within said hydrophobic solid component to provide the said hydrophobic solid component with mechanical stability and an enhanced dissolution after digestion for complete release of included probiotic bacteria, III. Optionally, an edible mediator, wherein said edible mediator is a glidant, anticaking and or lubricant.

Wherein the weight gain obtained by said first fatty coating layer (indicating the thickness of said first fatty coating layer) above 10%, preferable above 20% and most preferable above 25%.

Examples of edible hydrophobic solid component according to the present invention include fatty acids, fatty acid esters, fatty acid triesters, salts of fatty acids such as aluminum, sodium, potassium and magnesium, fatty alcohols, phospholipids, solid lipids, waxes, and a combination thereof wherein said edible hydrophobic solid component has a melting point above 20° C. and below 90° C., preferably above 25° C. and below 85° C. and most preferably above 30° C. and below 85° C.

Example of edible water-soluble polymeric stress absorber according to the present invention includes polymers selected from one of the group consisting of: I. Starches such as native starch, pregelatinized starch, partially gelatinized starch, hydrolyzed starch such as dextrin (E1400), acid-treated starch (E1401), alkaline-treated starch (E1402), bleached starch (E1403), oxidized starch (E1404), enzyme-treated starch (E1405), monostarch phosphate (E1410), distarch phosphate (E1412), phosphated distarch phosphate (E1413), acetylated distarch phosphate (E1414), starch acetate (E1420), acetylated distarch adipate (E1422), hydroxypropyl starch (E1440), hydroxypropyl distarch phosphate (E1442), hydroxypropyl distarch glycerol (E1443), starch sodium octenyl succinate (E1450), acetylated oxidized starch (E1451); II. Gums such as natural gums obtained from botanical resources such as arabic gum (E414), tragacanth gum (E413), karaya gum (E416), ghatti gum, guar gum (E412) from guar beans, carob (locust) bean gum (E410), konjac gum (E425), tara gum (E417), pectin and its derivatives, natural gums obtained from seaweeds such as agar (E406), alginic acid (E400) and sodium alginate (E401), carrageenan (E407), etc., natural gums produced by bacterial fermentation such as gellan gum (E418) and xanthan gum (E415); III. Cellulose ether (cellulose derivatives)—The commercially important properties of cellulose ethers are determined by their molecular weights, chemical structure and distribution of the substituent groups, degree of substitution and molar substitution (where applicable). These properties generally include solubility, viscosity in solution, surface activity, thermoplastic film characteristics and stability against biodegradation, heat, hydrolysis and oxidation. Viscosity of cellulose ether solutions is directly related with their molecular weights. Examples of mostly used cellulose ethers are: Methyl cellulose (MC), Ethyl cellulose (EC), Hydroxyethyl cellulose (HEC), Hydroxypropyl cellulose (HPC), hydroxypropylmethyl cellulose (HPMC), carboxymethyl cellulose (CMC) and sodium carboxymethyl cellulose (NaCMC); IV. vinyl polymers such as polyvinyl alcohol; V. and/or a combination thereof, wherein the weight percentage of said edible water-soluble polymeric stress absorber in said first fatty coating layer ranges between 1% and 20%, preferably between 2% and 15% and most preferably between 5% and 10%.

According to preferred embodiments of the present invention the first fatty coating layer may optionally contain an edible mediator, wherein said edible mediator is a glidant, anticaking and or lubricant such as sodium carbonate (E500), tricalcium phosphate (E341), potassium carbonate (E501), ammonium carbonate (E503), magnesium carbonate (E504), hydrochloric acid (E507), potassium chloride (E508), calcium chloride (E509), ammonium chloride (E510), magnesium chloride (E511), stannous chloride (E512), sulphuric acid (E513), sodium sulphates (E514), potassium sulphate (E515), calcium sulphate (E516), ammonium sulphate (E517), magnesium sulphate, epsom salts (E518), copper sulphate (E519), aluminium sulphate (E520), aluminium sodium sulphate (E521), aluminium potassium sulphate (E522), aluminium ammonium sulphate (E523), sodium hydroxide (E524), potassium hydroxide (E525), calcium hydroxide (E526), ammonium hydroxide (E527), magnesium hydroxide (E528), calcium oxide (E529), magnesium oxide (E530), sodium ferrocyanide (E535), potassium ferrocyanide (E536), calcium ferrocyanide (E538), dicalcium diphosphate (E540), sodium aluminium phosphate (E541), bone phosphate (E542), edible bone phosphate (derived from steaming animal bones and used as anti-caking agent, emulsifier and source of phosphorous in food supplements.), sodium silicate (E550), silicon dioxide (E551), calcium silicate (E552), (i) magnesium silicate and (ii) magnesium trisilicate (E553(a)), talc (E553(b)), sodium aluminum silicate (E554), potassium aluminum silicate (produced from several natural minerals.) (E555), aluminum calcium silicate (produced from several natural minerals.) (E556), bentonite (a natural type of clay from volcanic origin. it is a decolorising agent, filter medium, emulsifier and anti-caking agent. bentonite is used in pharmaceutical agents for external use, edible fats and oils, sugar, wine.) (E558), kaolin (E559), magnesium stearate, calcium stearate (E572), gluconic acid (E574), glucono delta-lactone (E575), sodium gluconate (E576), potassium gluconate (E577), calcium gluconate (E578), ferrous gluconate (E579), ferrous lactate (E585), polydimethylsiloxane (E900) and a combination thereof. Preferably calcium silicate, magnesium stearate, sodium aluminosilicate, talc, calcium stearate, bentonite and silicon dioxide.

According to some embodiments, the granule of the present invention includes low water activity (a w) due to the unique production process described herein. For example, the water activity in the resulting granule may be between 0.0001 and 0.3 a_{w}, preferably between 0-0.2 a_{w}.

According to some demonstrative embodiments, there is provided herein a process for the production of a probiotic granule, wherein the process includes:

forming a core using a hot melt process including:
I. Mixing probiotic bacteria with sugar particles, such as maltodextrin, trehalose, lactose, galactose, sucrose, fructose and the like, optionally adding a stabilizer such as oxygen scavenger containing L-cysteine base or hydrochloride;
optionally adding other food grade ingredients such as a filler, a surfactant, e.g., tween 80, and binder;
II. Melting at least one edible solid fat, wax or fatty acid;
III. Spraying the melt of said at least one edible solid fat, wax or fatty acid onto the mixture of said probiotic bacteria with sugar particles to form solid probiotic particles (the core);

coating the solid probiotic particles (the core) including:
I. Melting at least one an edible hydrophobic solid component having a melting point higher than 30° C., e.g., fat, wax or fatty acid, to form a hydrophobic film;
II. Adding and mixing at least one edible water-soluble polymeric stress absorber with the melt of said at least one an edible hydrophobic solid component to yield a melt of hydrophobic mixture; Optionally, adding an edible mediator, such as a glidant, anticaking and or lubricant to
III. Spraying the melt of the hydrophobic mixture onto the core to provide a probiotic granule;

Optionally, coating the probiotic granule with an enteric coating comprising an edible enteric polymer which may further provide protection through GI tract destructive parameters such as low pHs.

According to some embodiments, there is provided herein a use of the granule of the present invention in the preparation of a food product.

According to some embodiments, the food product may be selected from the group including grains such as wheat, rye, oats, corn, rice; grain products such as bakery goods, bread, rolls, cakes, cookies, pies, cereal, corn flakes, oat flakes, popcorn, pasta, macaroni, noodles, spaghetti; bread such as white bread, whole-wheat bread, rye bread, Rolls, buns, sesame roll, cinnamon roll, hamburger bun, hot dog bun, cakes, cookies and crackers; pastry such as pie, pizza, pancake, doughnut, muffin; meat and meat products; dairy products such as milk, yogurt, cream, sour cream, butter, cheese, ice cream; Sweets and chocolates such as candies, jelly candies, gummies, lollipops, chewing gums, caramels, chocolate candies, chocolate.

### EXAMPLES

### Example 1

A. Microencapsulated Bacteria for Supplement Products

1. The Preparation of the Microcapsules (Microencapsulation Process)

The microcapsules were formed first by the creation of the core and then coating of the core by at least one coating layer.

The core was formed by a granulation process through which the particles of the bacteria and the filler were combined by spraying the binder (a melt of a hydrophobic component such as a fatty acid) to form particles on which the coating layers could further be applied. The coating layer was performed by spraying a melt of a hydrophobic component under specific coating conditions which could allow the formation of a uniform film coating surrounding the core. The microencapsulation of BL was performed based on a fluid bed method, using a Romaco Innojet VENTILUS® V2.5 systems (Romaco Innojet, Steinen, Germany).

An example of the components is presented in table 1

TABLE 1

| | | | | % w/w |
|---|---|---|---|---|
| | | | | in final |
| Material | | | | micro- |
| Name | Product Name | Supplier | Use | capsule |
| Probiotic | | | Active | 50 |
| bacteria | | | ingredient | |
| Maltodextrin | MALDEX G190 | TEROSE | Filler | 24 |
| | PHARMA | SYRAL | | |
| Stearic Acid | Stearic acid | Emery | Binder, | 23.85 |
| | | Oleochemicals | Coating | |
| | | | agent | |
| High | Empure ® ES | EMSLAND | Shock- | 1.55 |
| amylose | 200 | GROUP | absorbing | |
| starch | | | agent | |
| Fumed silica | Aerosil 200 | Evonik | Flow aid | 0.55 |
| | Pharma | | | |

The microcapsules were prepared first by creating a core containing the probiotic bacteria followed by coating the core with at least one protective layer. The core was prepared by a melt granulation method. First, stearic acid (SA) was melted in a container by heating it to 85° C. using a heating plate. After the SA was fully melted, it was sprayed onto a well premixed freeze-dried probiotic powder and maltodextrin to consolidate the particles to form agglomerates as the core containing filler, binder and the bacteria. Separately, a mixture of High amylose starch (Empure® ES 200 is an example of a specific trademark of Emsland Group (Germany) for a clean label pea starch which is a high amylose starch, with adjusted swelling and dissolving properties for the food industry; it was used as binding, thickening and shock-absorbing agent) and Fumed silica (Aerosil® for example, is a trademark of Evonik (Germany) for hydrophilic fumed silica which was used as an anti-caking aid and flow aid) was added to the melted SA while mixing and keeping on the heating plate set at 85° C. The resulting melt combination was then sprayed onto the resulting core (granules) to reach an appropriate weight gain (% w/w) in relation to the initial core weight. The thickness of the layers was expressed by the % weight gain (WG) which was obtained upon the coating process in relative to the initial substrate's weight prior to the coating process according to the following equation:

$$\% WG = \frac{WG}{W 0} \times 100 = \frac{Wd - W0}{W 0} \times 100$$

Where Wd and W0 are respectively the weight of the substrate after and before coating process and WG is the weight gain.

2. Parker Test

Test Procedure:

Alu/Alu sachet packs containing 2.0 g of powder [either free bacteria (uncoated bacteria) or microencapsulated bacteria or microcapsules+maltodextrin] were exposed to constant temperature of 37° C. in an incubator [Lab. Incubator, MRC Israel] (controlled humidity) for 28 days. Packs were taken in 7, 14, 21, 28 days for microbiology tests to determine the stability of the bacteria.

Prior to enumeration test the samples were pretreated to release the available bacteria from the microcapsules according to the following method:

3. Method for Releasing the Bacteria from the Microcapsules Prior to Enumeration Test

First a sample of 2-g microencapsulated bacteria was accurately weighed. The sample was gently dispersed into 8 ml purified water (in a 20 ml volume glass vial with a cap) and shaked for 60 minutes using a water-bath shaker set at 37° C.

The resulting dispersion was then stirred by a Vortex for 20-30 seconds and gently poured into a stomacher bag. The residues left in the vial were washed with 10 ml peptone solution (pH 7.3) and added into the stomacher bag; this makes the first dilution (1:10 dilution) in the stomacher bag. The content in the bag was manually mixed and subsequently stomached for 1 minute using a stomacher. The sample was then ready for a series of dilutions and performance of viable cell count according to the bacteria species and strain.

### Example A1—Lactobacillus johnsonii 456

TABLE 2

| | | WG % | % | Results [CFU/g bacteria] | | | | |
|---|---|---|---|---|---|---|---|---|
| Batch No. | Formula | Coating | Bacteria | Time 0 | 7 d | 14 d | 21 d | 28 d |
| Free | — | — | — | 1.8 × 10¹¹ | 1.2 × 10¹¹ | 1.6 × 10¹¹ | 8.0 × 10¹⁰ | 2.5 × 10¹⁰ |
| L. Johnsonii | | | | | | | | |
| [13072020] | Stearic Acid | 20 | 47.5 | 1.6 × 10¹¹ | 8.1 × 10¹⁰ | 1.35 × 10¹¹ | 1.2 × 10¹¹ | 1.4 × 10¹¹ |
| [12082020] | Stearic Acid | 30 | 43.1 | 1.3 × 10¹¹ | 1.8 × 10¹¹ | 1.36 × 10¹¹ | 1.7 × 10¹¹ | 1.3 × 10¹¹ |
| [28122020] | [92:6:2]* | 35 | 68.3 | 2.34 × 10¹¹ | 1.9 × 10¹¹ | 1.4 × 10¹¹ | 1.6 × 10¹¹ | 2.34 × 10¹¹ |
| | SA:ES200:Aerosil | | | | | | | |
| [28122020] + | [92:6:2] | 35 | 34.15 | 4.8 × 10¹¹ | 1.65 × 10¹¹ | 2.9 × 10¹¹ | 2.9 × 10¹¹ | 1.55 × 10¹¹ |
| M | SA:ES200:Aerosil | | | | | | | |
| [28122020] | [92:6:2] | 35 | 68.3 | 1.5 × 10¹¹ | 1.8 × 10¹¹ | 9.5 × 10¹⁰ | 1.6 × 10¹¹ | 1.1 × 10¹¹ |
| | SA:ES200:Aerosil | | | | | | | |
| [28122020] + | [92:6:2] | 35 | 34.15 | 2.5 × 10¹¹ | 2.4 × 10¹¹ | 1.5 × 10¹¹ | 1.4 × 10¹¹ | 1.7 × 10¹¹ |
| M | SA:ES200:Aerosil | | | | | | | |

SA—Stearic acid
M—maltodextrin
Aerosil ® is a trademark of Evonik (Germany) for hydrophilic fumed silica which was used as an anti-caking aid and flow aid
Empure ® ES 200 is a specific trademark of Emsland Group (Germany) for a clean label pea starch (high amylose starch), with adjusted swelling and dissolving properties for the food industry; it was used as binding, thickening and shock-absorbing agent
*The weight ratio between SA, ES200 and Aerosil respectively
Time 0 indicates the viability of the bacteria (for microencapsulated bacteria right after microencapsulation process) before exposure to 37° C.

### Example 2.1—Lactobacillus Rhamnosus

TABLE 3

| | | WG % | % | Results [CFU/g Bacteria] | | | | |
|---|---|---|---|---|---|---|---|---|
| | Formula | Coating | Bacteria | Time 0 | 7 d | 14 d | 21 d | 28 d |
| Free | — | — | — | 3.2 × 10¹¹ | 2.1 × 10¹¹ | 8 × 10¹⁰ | 1.1 × 10¹⁰ | 1.9 × 10⁹ |
| L. | | | | | | | | |
| Rhamnosus | | | | | | | | |
| BIO | | | | | | | | |
| [Belgium] | | | | | | | | |
| Free | — | — | — | 3.2 × 10¹¹ | 1.1 × 10¹¹ | 2.6 × 10¹⁰ | 1.5 × 10¹⁰ | 2.1 × 10⁹ |
| L. | | | | | | | | |
| Rhamnosus | | | | | | | | |
| [France] | | | | | | | | |
| [22062020] | Pure stearic acid | 20 | 47.5 | 4 × 10¹¹ | 4 × 10¹¹ | 8.4 × 10¹⁰ | 3.2 × 10¹⁰ | 1 × 10¹⁰ |
| [20072020] | Pure cocoa butter | 20 | 40 | 1.4 × 10¹¹ | 1.3 × 10¹¹ | 5 × 10¹⁰ | 9.9 × 10⁹ | 1 × 10⁶ |
| [France] | | | | | | | | |
| [20072020] | Pure lauric acid | 44 | 21.5 | 0 | 0 | — | — | — |
| [28072020] | Pure meristic | 40 | 21.5 | 2.5 × 10⁹ | — | 8.1 × 10⁵ | — | — |
| | acid | | | | | | | |
| [09092020] | [95:2.5:2.5]* | 20 | 39 | 1 × 10¹¹ | 1.1 × 10¹¹ | 2.6 × 10¹⁰ | 1.5 × 10¹⁰ | 2.1 × 10⁹ |
| [France] | SA:Emfix:HPS | | | | | | | |
| (0.04) | | | | | | | | |
| [09092020] | [95:2.5:2.5] | 20 | 39 | 1 × 10¹¹ | 5.6 × 10⁹ | 5.6 × 10⁷ | 5.6 × 10⁶ | 1.6 × 10⁵ |
| [France] | SA:Emfix:HPS | | | | | | | |
| (0.01) | | | | | | | | |
| [09092020] | [95:2.5:2.5] | 20 | 19.5 | 1 × 10¹¹ | 3.8 × 10¹⁰ | 2 × 10⁸ | 2.2 × 10⁸ | 2.95 × 10⁷ |
| [France] + | SA:Emfix:HPS | | | | | | | |
| M | | | | | | | | |
| (0.00) | | | | | | | | |
| [22092020] | [93:5:2] | 20 | 46 | 7.9 × 10¹⁰ | 2.1 × 10¹⁰ | 7.2 × 10⁸ | 2.8 × 10⁸ | 5.3 × 10⁷ |
| [France] | SA:Emfix:HPS | | | | | | | |
| [22092020] | [93:5:2] | 20 | 46 | 9.7 × 10¹⁰ | 2.2 × 10¹⁰ | 7.3 × 10⁸ | 4.4 × 10⁸ | 1.7 × 10⁸ |
| [France] | SA:Emfix:HPS | | | | | | | |
| [22092020] | [93:5:2] | 20 | 23 | 1.2 × 10¹¹ | 8.7 × 10¹⁰ | 8 × 10⁸ | 1.1 × 10¹⁰ | 6.5 × 10⁹ |
| [France] + | SA:Emfix:HPS | | | | | | | |
| M | | | | | | | | |
| [21122020] | [92:6:2] | 35 | 73.1 | 3.8 × 10¹¹ | 2.75 × 10¹¹ | 6.7 × 10⁹ | 3.1 × 10⁸ | 1.37 × 10⁸ |
| | SA:ES200:Aerosil | | | | | | | |
| [21122020] + | [92:6:2] | 35 | 36.6 | 3.9 × 10¹¹ | 5.3 × 10¹¹ | 4.64 × 10¹¹ | 1.05 × 10¹¹ | 4.7 × 10¹⁰ |
| M | SA:ES200:Aerosil | | | | | | | |
| [21122020] + | [92:6:2] | 35 | 50 | 3.9 × 10¹¹ | 1 × 10¹¹ | 5.8 × 10¹⁰ | 2.2 × 10¹⁰ | 2.2 × 10¹⁰ |
| M | SA:ES200:Aerosil | | | | | | | |
| [18012021] | [92:6:2] | 20 | 46 | 3.7 × 10¹¹ | 1.4 × 10¹¹ | 9.6 × 10¹⁰ | 6.5 × 10¹⁰ | 8.5 × 10¹⁰ |
| | SA:ES200:Aerosil | | | | | | | |
| [1801202]1 + | [92:6:2] | 20 | 23 | 3.7 × 10¹¹ | 2.6 × 10¹¹ | 4.1 × 10¹¹ | 4 × 10¹¹ | 3.8 × 10¹¹ |
| M | SA:ES200:Aerosil | | | | | | | |
| [19012021] | [92:8] | 20 | 46 | 4 × 10¹¹ | 1 × 10¹¹ | 4.8 × 10¹⁰ | 1.2 × 10¹¹ | 1 × 10¹¹ |
| | SA:ES200 | | | | | | | |
| [19012021] + | [92:8] | 20 | 23 | 5 × 10¹¹ | 3.1 × 10¹¹ | 1.7 × 10¹¹ | 2 × 10¹¹ | 3 × 10¹¹ |
| M | SA:ES200 | | | | | | | |
| [10022021] | [92:6:2] | 35 | 70 | 2.8 × 10¹¹ | 1.6 × 10¹¹ | 2.86 × 10¹⁰ | 3.9 × 10¹⁰ | 1.4 × 10⁹ |
| | SA:HPS:Aerosil | | | | | | | |
| [10022021] + | [92:6:2] | 35 | 35 | 5.5 × 10¹¹ | 2 × 10¹¹ | 3.4 × 10¹¹ | 3.2 × 10¹¹ | 1 × 10¹¹ |
| M | SA:HPS:Aerosil | | | | | | | |
| [16022021] | [92:6:2] | 35 | 70 | 3.6 × 10¹¹ | 3.6 × 10⁹ | 2.5 × 10⁸ | 5.7 × 10⁷ | 2.6 × 10⁸ |
| | SA:Emfix:Aerosil | | | | | | | |
| [16022021] + | [92:6:2] | 35 | 35 | 3.1 × 10¹¹ | 1.4 × 10¹¹ | 2 × 10¹⁰ | 1 × 10¹¹ | 6.2 × 10¹⁰ |
| M | SA:Emfix:Aerosil | | | | | | | |
| [15062020] | [92:6:2] | 20 | 50 | 1.1 × 10¹¹ | 2.6 × 10¹⁰ | 4.4 × 10¹⁰ | 2.4 × 10¹⁰ | 6.6 × 10¹⁰ |
| | SA:ES200:Aerosil | | | | | | | |
| [15062020] + | [92:6:2] | 20 | 25 | 1.2 × 10¹¹ | 1.8 × 10¹¹ | 4 × 10¹¹ | 3 × 10⁸ | 1.48 × 10¹¹ |
| M | SA:ES200:Aerosil | | | | | | | |

*The weight ratio between the components of the coating layer respectively
Time 0 indicates the viability of the bacteria (for microencapsulated bacteria right after microencapsulation process) before exposure to 37° C.

### Example 2.2—Lactobacillus Rhamnosus GG (Chr. Hansen)

TABLE 4

| Sample | | WG % | % | | Results [CFU/g Bacteria] | | | | |
|---|---|---|---|---|---|---|---|---|---|
| Type | Formula | Coating | Bacteria | Aw | Time 0 | 7 d | 14 d | 21 d | 28 d |
| Free L. | — | — | — | 0.015 | 3.2 × 10¹¹ | 2 × 10¹⁰ | 2.5 × 10¹⁰ | 2 × 10¹⁰ | 7.2 × 10¹⁰ |
| Rhamnosus | | | | | | | | | |
| [Chr. Hansen] | | | | | | | | | |
| [16112020] | [92:6:2]* | 35 | 20.4 | 0.008 | 3.3 × 10¹¹ | 3.5 × 10¹¹ | 3.4 × 10¹¹ | 3.5 × 10¹¹ | 3.5 × 10¹¹ |
| | SA:ES200:Aerosil | | | | | | | | |
| [16112020] + | [92:6:2] | 35 | 10.2 | 0.008 | 3.3 × 10¹¹ | 3.4 × 10¹¹ | 3.5 × 10¹¹ | 3.5 × 10¹¹ | 3.5 × 10¹¹ |
| M | SA:ES200:Aerosil | | | | | | | | |

*The weight ratio between the components of the coating layer respectively
Time 0 indicates the viability of the bacteria (for microencapsulated bacteria right after microencapsulation process) before exposure to 37° C.

### Example 2.3—Lactobacillus Rhamnosus (Glac Probiotics)

TABLE 5

| Sample | | WG % | % | | Results [CFU/g Bacteria] | | | | |
|---|---|---|---|---|---|---|---|---|---|
| Type | Formula | Coating | Bacteria | Aw | Time 0 | 7 d | 14 d | 21 d | 28 d |
| Free | — | — | — | 0.015 | 3.5 × 10¹¹ | 2 × 10¹¹ | 1.1 × 10¹¹ | 7 × 10¹⁰ | 2.1 × 10¹⁰ |
| L. | | | | | | | | | |
| Rhamnosus | | | | | | | | | |
| [18102020] | [92:5:3]* | 22.5 | 76.3 | 0.008 | 3.9 × 10¹¹ | 5.2 × 10¹⁰ | 6.2 × 10⁹ | 3.6 × 10⁵ | 3.5 × 10⁷ |
| | SA:Emfix:Aerosil | | | | | | | | |
| [18102020] | [92:5:3] | 22.5 | 76.3 | 0.008 | 4 × 10¹¹ | 3.3 × 10¹¹ | 7.8 × 10¹⁰ | 1.8 × 10¹⁰ | 5.6 × 10⁸ |
| Dried | SA:Emfix:Aerosil | | | | | | | | |
| [18102020] + | [92:5:3] | 22.5 | 38.15 | 0.000 | 4 × 10¹¹ | 2.67 × 10¹¹ | 8.5 × 10¹⁰ | 1 × 10¹¹ | 4.4 × 10¹⁰ |
| M | SA:Emfix:Aerosil | | | | | | | | |
| [19102020] | Layer 1: SA | 27.4 | 73.4 | 0.012 | 3.6 × 10¹¹ | 1.56 × 10¹⁰ | 1.5 × 10⁸ | 2.1 × 10⁷ | 1.4 × 10⁷ |
| | Layer 2: [93:5:2] | | | | | | | | |
| | SA:Emfix:Aerosil | | | | | | | | |
| [19102020] | Layer 1: SA | 27.4 | 73.4 | 0.000 | 3.4 × 10¹¹ | 1 × 10¹¹ | 2.2 × 10¹⁰ | 9.9 × 10⁷ | 2.45 × 10⁸ |
| Dried | Layer 2: [93:5:2] | | | | | | | | |
| | SA:Emfix:Aerosil | | | | | | | | |
| [19102020] + | Layer 1: SA | 27.4 | 36.7 | 0.000 | 3.9 × 10¹¹ | 2.7 × 10¹¹ | 8.7 × 10¹⁰ | 5.44 × 10⁹ | 3.26 × 10¹⁰ |
| M | Layer 2: [93:5:2] | | | | | | | | |
| | SA:Emfix:Aerosil | | | | | | | | |
| [20122020] | [92:6:2] | 35 | 77 | 0.00 | 4.1 × 10¹¹ | 2.3 × 10¹¹ | 1.35 × 10¹¹ | 2.0 × 10¹⁰ | 1.25 × 10¹⁰ |
| Dried | SA:ES200:Aerosil | | | | | | | | |
| [20122020] + | [92:6:2] | 35 | 38.5 | 0.00 | 5.2 × 10¹¹ | 4.8 × 10¹¹ | 1.35 × 10¹¹ | 5.9 × 10¹⁰ | 4 × 10¹⁰ |
| M | SA:ES200:Aerosil | | | | | | | | |

*The weight ratio between the components of the coating layer respectively
Time 0 indicates the viability of the bacteria (for microencapsulated bacteria right after microencapsulation process) before exposure to 37° C.

### Example 3.1—Bifidobacterium Lactis (Glac Probiotics)

TABLE 6

| Sample | | WG % | % | Results [CFU/g Bacteria] | | | | |
|---|---|---|---|---|---|---|---|---|
| Type | Formula | Coating | Bacteria | Time 0 | 7 d | 14 d | 21 d | 28 d |
| Free B. | — | — | — | 2.6 × 10¹¹ | 2 × 10¹¹ | 1.1 × 10¹¹ | 1.2 × 10¹¹ | 8.3 × 10¹⁰ |
| Lactis | | | | | | | | |
| [06072020] | 100% Stearic Acid | 22.2 | 43.1 | 2 × 10¹¹ | 2 × 10¹¹ | 1.7 × 10¹¹ | 1.4 × 10¹¹ | 8 × 10¹⁰ |
| [29122020] | [92:6:2]* | 35 | 70.7 | 2.48 × 10¹¹ | 2.9 × 10¹¹ | 1.55 × 10¹¹ | 2.4 × 10¹¹ | 1.45 × 10¹¹ |
| | SA:ES200:Aerosil | | | | | | | |
| [29122020] + | [92:6:2] | 35 | 35.35 | 5.8 × 10¹¹ | 6 × 10¹¹ | 6.8 × 10¹¹ | 4.5 × 10¹¹ | 2.85 × 10¹¹ |
| M | SA:ES200:Aerosil | | | | | | | |

*The weight ratio between the components of the coating layer respectively
Time 0 indicates the viability of the bacteria (for microencapsulated bacteria right after microencapsulation process) before exposure to 37° C.

### Example 3.2—Bifidobacterium Lactis VES002

TABLE 7

| Sample | | WG % | % | Results [CFU/g Bacteria] | | | | |
|---|---|---|---|---|---|---|---|---|
| Type | Formula | Coating | Bacteria | Time 0 | 7 d | 14 d | 21 d | 28 d |
| Free B. | — | — | — | 2.1 × 10¹¹ | 1.75 × 10¹¹ | 6.8 × 10¹⁰ | 5.9 × 10¹⁰ | 1.6 × 10¹⁰ |
| Lactis | | | | | | | | |
| [18082020] | 100% Stearic Acid | 27 | 40 | 4.2 × 10¹¹ | 1.64 × 10¹¹ | 8 × 10¹⁰ | 1.64 × 10¹¹ | 1.5 × 10¹¹ |
| [22122020] | [92:6:2]* | 35 | 70.7 | 5 × 10¹¹ | 2.4 × 10¹¹ | 4.2 × 10¹¹ | 3.35 × 10¹¹ | 2.25 × 10¹¹ |
| | SA:ES200:Aerosil | | | | | | | |
| [22122020] + | [92:6:2] | 35 | 35.35 | 5 × 10¹¹ | 4.5 × 10¹¹ | 6.1 × 10¹¹ | 6.1 × 10¹¹ | 5.8 × 10¹¹ |
| M | SA:ES200:Aerosil | | | | | | | |

*The weight ratio between the components of the coating layer respectively
Time 0 indicates the viability of the bacteria (for microencapsulated bacteria right after microencapsulation process) before exposure to 37° C.

### Example 4—Bifidobacterium Breve M-16V (Morinaga)

TABLE 8

| Sample | | WG % | % | | Results [CFU/g Bacteria] | | | | |
|---|---|---|---|---|---|---|---|---|---|
| Type | Formula | Coating | Bacteria | Aw | Time 0 | 7 d | 14 d | 21 d | 28 d |
| Free B. | — | — | — | 0.018 | 1.6 × 10¹¹ | 1.8 × 10¹¹ | 1.8 × 10¹¹ | 1.85 × 10¹¹ | 1.9 × 10¹¹ |
| Breve | | | | | | | | | |
| [Morinaga] | | | | | | | | | |
| [23112020] | [90:8:2]* | 35 | 20 | 0.02 | 2 × 10¹¹ | 1.5 × 10¹¹ | 1.6 × 10¹¹ | 1.9 × 10¹¹ | 2 × 10¹¹ |
| Dried | SA:ES200:Aerosil | | | | | | | | |
| [23112020] + | [90:8:2] | 35 | 10 | 0.008 | 2 × 10¹¹ | 2 × 10¹¹ | 2.8 × 10¹¹ | 1.9 × 10¹¹ | 2.3 × 10¹¹ |
| M | SA:ES200:Aerosil | | | | | | | | |

*The weight ratio between the components of the coating layer respectively
Time 0 indicates the viability of the bacteria (for microencapsulated bacteria right after microencapsulation process) before exposure to 37° C.

### Example 5—Bifidobacterium Longum

TABLE 9

| Sample | | WG % | % | Results [CFU/g Bacteria] | | | | |
|---|---|---|---|---|---|---|---|---|
| Type | Formula | Coating | Bacteria | Time 0 | 7 d | 14 d | 21 d | 28 d |
| Free B. | — | — | — | 9.2 × 10¹⁰ | 8.3 × 10⁸ | 5.3 × 10⁸ | 1 × 10⁸ | 1.2 × 10⁷ |
| Longum | | | | | | | | |
| [10112020] | [91:7:2]* | 25.8 | 50.2 | 3.8 × 10¹⁰ | 5.3 × 10⁷ | 2 × 10⁸ | 2.44 × 10⁷ | 1.7 × 10⁷ |
| | SA:Emfix:Aerosil | | | | | | | |
| [10112020] | [91:7:2] | 25.8 | 50.2 | 4.8 × 10¹⁰ | 1.6 × 10⁹ | 2.45 × 10⁹ | 1.95 × 10⁹ | 6 × 10⁹ |
| Dried | SA:Emfix:Aerosil | | | | | | | |
| [10112020] + | [91:7:2] | | | | | | | |
| Maltodextrin | SA:Emfix:Aerosil | 25.8 | 25.1 | 3 × 10¹⁰ | 2.3 × 10⁹ | 1.15 × 10¹⁰ | 4 × 10⁹ | 1.1 × 10¹⁰ |

*The weight ratio between the components of the coating layer respectively
Time 0 indicates the viability of the bacteria (for microencapsulated bacteria right after microencapsulation process) before exposure to 37° C.

Stability Tests at High Temperatures

For this stability test microencapsulation process according to the present invention was performed on Lacticaseibacillus rhamnosus (L. rhamnosus) using different formulations as presented in Table 10 and the viability of the bacteria was then determined and compared to that of the free bacteria.

The bacteria were released from the microcapsules by shaking the vial containing the sample using a vortex for 20 seconds to disperse the sample followed by vigorous shaking by hand for 40 seconds and finally shaking by vortex for additional 10 seconds.

Enumeration Test was Performed as Follows:

The serial decimal dilutions were done with TS+[Tryptone solution (TS+) which was prepared by dissolving tryptone (15.0 g) and sodium chloride (8.5 g) in distilled or deionized water, while warming gently, and filling to a final volume of 1000 ml. Tween 80 was then added (1 mL) to obtain an end-concentration of 0.001%. The final solution was finally sterilized by heat treatment of 121±1° C. for 15±1 min. The pH of the solution was measured 7.0±0.2 (at 25±1° C.). A pour-plate method was used to count the number of live LR. To this end, 1 mL of the appropriate dilutions was transferred to Petri dishes and mixed with 15-20 mL of MRS agar (properly prepared, according to the manufacturer's instructions, and autoclaved) containing 0.5% of filter sterilized L-cysteine hydrochloride solution (10% in distilled water). The inoculated agar plates were then left on a cool even surface to solidify. After solidification, the plates were inverted and subsequently incubated anaerobically in a jar with the Anaerogen (Oxoid, Hampshire, UK) system, for 72±4 h at 37° C. The plates with 30-300 colonies were then used for calculating the colony-forming unit (CFU) per 1 g of the sample (CFU/g).

TABLE 10
Viable cell count of Lacticaseibacillus rhamnosus right after microencapsulation
process (20% w/w bacteria content) based on 35% w/w coating layer with different
formulations as compared to that of the free bacteria (uncoated bacteria)
CFU/g Bacteria

| | [04012021]* | [25012021] | [22022021] | [28022021]^{a} | [25052021] | [30062021] | [13072021]^{b} |
|---|---|---|---|---|---|---|---|
| Free | [92:6:2] | [92:8] | [100] | [92:6:2] | [86:10:4] | [92:6:2] | [92:6:2] |
| Bacteria | SA:ES200:Aerosil | SA:ES200 | SA | SA:ES200:Aerosil | SA:ES200:Aerosil | SA:ES200:Aerosil | SA:ES200:Aerosil |
| 3.2 × 10¹¹ | 3.3 × 10¹¹ | 4 × 10¹¹ | 5.5 × 10¹¹ | 5 × 10¹¹ | 1.1-1.7 × 10¹¹ | 4.4 × 10¹¹ | 4.3 × 10¹¹ |

^{a}Core containing lactose
^{b}45% w/w coating layer
SA—Stearic acid
Aerosil ® is a trademark of Evonik (Germany) for hydrophilic fumed silica which was used as an anti-caking aid and flow aid.
Empure ® ES 200 is a specific trademark of Emsland Group (Germany) for a clean label pea starch (high amylose starch), with adjusted swelling and dissolving properties for the food industry; it was used as binding, thickening and shock-absorbing agent.
*The weight ratio between the components of the coating layer respectively

### Example 6—Stability in Water at 72-75° C.

Test Procedure:

Microencapsulated Lacticaseibacillus rhamnosus (L. rhamnosus) (either 0.002 or 0.02 g) was dispersed in 10 mL distilled water at 72-75° C. for 15 seconds and subsequently cooled in an ice water bath for 60 seconds. The viability of the bacteria was then compared to that of the free bacteria exposed to the same conditions. The results are presented in Table 11 as follows:

TABLE 11
Viability of the bacteria after exposure
to water at 72-75° C. for 15 sec.

| | | Bacteria/ | |
|---|---|---|---|
| Batch | | Microcapsules | CFU/g Bacteria |
| No. | Coating Formula | Quantity [g] | 15 sec at 72-75° C. |
| Free bacteria | | 0.002 | 1.25 × 10⁹ |
| [22022021] | [100] SA | 0.002 | 3.2 × 10⁸ |
| [04012021] | [92:6:2]* | 0.002 [1] | 1.7 × 10¹⁰ |
| | SA:ES 200:Aerosil | 0.002 [2] | 6.3 × 10¹⁰ |
| | | 0.02 | 4 × 10¹⁰ |
| [25012021] | [92:8] | 0.002 [1] | 6.4 × 10¹⁰ |
| | SA:ES 200 | 0.002 [2] | 4.1 × 10¹⁰ |
| | | 0.02 | 3.9 × 10¹⁰ |

*The weight ratio between the components of the coating layer respectively

### Example 7—Stability in water at 42° C. followed by 72-75° C.

Test Procedure:

Microencapsulated Lacticaseibacillus rhamnosus (L. rhamnosus) (either 0.002 or 0.02 g) was gently dispersed in distilled water at 42° C. and retained for 240 min with no shaking (Stage I). The temperature was then raised to 73-74° C. using a water circulating bath (it took about 1 minute to reach 73-74° C. from 42° C.) and the samples were retained at this temperature for 15 sec. with no shaking (but under continuous circulation of water) (Stage II). The dispersion was then cooled for 60 sec. in an ice water bath with no shaking. The viability of the bacteria was then determined after each step and compared to that of the free bacteria exposed to the same conditions. The results are presented in Table 12 as follows:

TABLE 12

| | | Bacteria/ | | |
|---|---|---|---|---|
| | | Micro- | | |
| | | capsules | | |
| | | Quantity | CFU/g Bacteria | |
| Batch No. | Coating Formula | [g] | Stage I | Stage II |
| Free bacteria | | 0.002 | 3.3 × 10¹¹ | 1 × 10⁴ |
| [22022021] | [100] SA | 0.002 | 5 × 10¹¹ | 4.2 × 10¹⁰ |
| [04012021] | [92:6:2]* | 0.002 [1] | 5 × 10¹¹ | 3.3 × 10¹¹ |
| | SA:ES 200:Aerosil | 0.002 [2] | 6 × 10¹¹ | 2.5 × 10¹¹ |
| | | 0.02 | 3.5 × 10¹¹ | 1.8 × 10¹¹ |
| [25012021] | [92:8] | 0.002 [1] | 7 × 10¹¹ | 5 × 10¹¹ |
| | SA:ES 200 | 0.002 [2] | 5 × 10¹¹ | 2.4 × 10¹¹ |
| | | 0.02 | 3 × 10¹¹ | 1.5 × 10¹⁰ |

*The weight ratio between the components of the coating layer respectively

### Example 8—a One-Week Shelf Stability of the Samples from Stage II in Water at Different Temperatures

Test Procedure:

Samples from Example 7, Stage II (dispersion in water) were taken for shelf-life stability by storing at 4-6° C. and at Room Conditions for 1 week. The viability of the bacteria was then determined for each temperature and compared to that of the free bacteria exposed to the same conditions. The results are presented in Table 13 as follows:

TABLE 13
Viability of the samples from Stage II (dispersion in water)
after storing at 4-6° C. and at Room Conditions for 1 week.

| | | Bacteria/ | Results [CFU/g Bacteria] | | |
|---|---|---|---|---|---|
| Test | Coating | Microcapsules | Right after | Room | |
| Sample | Formula | Quantity [g] | Stage II | Conditions | 4-6° C. |
| Free | | 0.002 | 1 × 10⁴ | 1.3 × 10⁴ | 4.6 × 10⁷ |
| bacteria | | | | | |
| [22022021] | [100] SA | 0.002 | 4.2 × 10¹⁰ | 2.3 × 10¹⁰ | 5.4 × 10¹⁰ |
| [04012021] | [92:6:2]* | 0.002 [1] | 3.3 × 10¹¹ | 3.25 × 10¹¹ | 4 × 10¹¹ |
| | SA:ES | 0.002 [2] | 2.5 × 10¹¹ | 7.3 × 10¹⁰ | 2.6 × 10¹¹ |
| | 200:Aerosil | 0.02 | 1.8 × 10¹¹ | 6.9 × 10¹⁰ | 2.55 × 10¹¹ |
| [25012021] | [92:8] | 0.002 [1] | 5 × 10¹¹ | 2.5 × 10¹⁰ | 6.25 × 10¹⁰ |
| | SA:ES 200 | 0.002 [2] | 2.4 × 10¹¹ | 4.5 × 10¹⁰ | 8.9 × 10¹⁰ |
| | | 0.02 | 1.5 × 10¹⁰ | 5 × 10⁹ | 2.2 × 10¹⁰ |

*The weight ratio between the components of the coating layer respectively

### Example 9—Stability in Milk at 72-75° C. (Pasteurization Process)

### Test Procedure

First a certain quantity of microcapsules was accurately weighed and properly dispersed in 45 mL cow milk (3% fat) (40 mL for dispersion and additional 5 mL later for washing the residues left in the stomacher bag) in a stainless steel Turkish warmer coffee pot-Finjan at room temperature using a laboratory spoon for a few seconds. The resulting dispersion was heated to 72° C. using a water bath (it took 70-75 seconds to reach the target temperature). The dispersion was then retained at 72° C. for 15 seconds (simulated pasteurization process) and immediately thereafter cooled in an ice bath for 30 sec. The samples were further treated first by a homogenizer for 30 sec at 5000 RPM followed by a stomacher (in a stomacher bag) for 120 Sec prior to the serial dilution and enumeration test. The viability of the bacteria was then determined and compared to that of the free bacteria exposed to the same conditions. The results are presented in Table 14 as follows:

TABLE 14
Viability of the bacteria after exposure to cow milk (3% fat) at 72-75° C.

| | | | | Pasteurization | Pasteurization |
|---|---|---|---|---|---|
| | | Bacteria/ | | (15 sec at 72° C.) | (30 sec at 72° C.) |
| Batch | | Microcapsules | | CFU per 1 g | CFU per 1 g |
| No. | Test Type | Quantity [g] | Control* | bacteria | bacteria |
| Free bacteria | Homogenizer | 5 | 3.2 × 10¹¹ | 2.75 × 10⁹ | — |
| [04012021] | Pasteurization + | 0.01 | 3.3 × 10¹¹ | 2.5 × 10¹¹ | — |
| | Homogenizer + | | | | |
| | Stomacher | | | | |
| | Pasteurization + | 5 | | 1.0 × 10¹¹ | 1.7 × 10¹¹ |
| | Homogenizer + | | | 3.2 × 10¹¹ | |
| | Stomacher | | | | |
| [04012021] | | 0.05 | | 5 × 10⁹ | — |
| 20% w/w | Pasteurization + | 5 | | 1 × 10¹⁰ | |
| coating | Homogenizer + | | | | |
| | Stomacher | | | | |
| [04012021] | Pasteurization + | 5 | | 8.1 × 10¹⁰ | — |
| 45% w/w | Homogenizer + | | | | |
| coating | Stomacher | | | | |
| [25012021] | Pasteurization + | 0.01 | 4.0 × 10¹¹ | 1.0 × 10¹¹ | 1.6 × 10¹¹ |
| | Homogenizer + | | | | |
| | Stomacher | | | | |
| | Pasteurization + | 5 | | 1.2 × 10¹¹ | — |
| | Homogenizer + | | | 1.8 × 10¹¹ | |
| | Stomacher | | | | |
| [25052021] | Pasteurization + | 0.01 | | | 7.5 × 10¹⁰ |
| | Homogenizer + | 0.05 | | | 7.7 × 10¹⁰ |
| | Stomacher | 5 | | | 4.5 × 10¹¹ |
| | Pasteurization + | 5 | | 1.5 × 10¹¹ | — |
| | Stomacher | | | | |
| [28022021] | Pasteurization | 0.01 | 5.0 × 10¹¹ | 3.7 × 10¹¹ | — |
| | Pasteurization + | 0.01 | | 4.0 × 10¹¹ | — |
| | Homogenizer + | | | | |
| | Stomacher | | | | |

*The control was the sample before simulated pasteurization process

### Example 10—Stability of Samples Treated by Homogenizer Followed by a Simulated Pasteurization Process (in Milk at 72-75° C.)

Test Procedure:

The sample was properly dispersed in 45 mL (40 mL for dispersion and additional 5 mL later for washing the residues left in the stomacher bag) milk (3% fat) (in a stainless steel Turkish warmer coffee pot-Finjan) at room temperature using a laboratory spoon for a few seconds followed by a homogenizer for 30 sec at 5000 RPM. The resulting dispersion was heated to 72° C. using a water bath (it took 70-75 seconds to reach the target temperature). The dispersion was then retained at 72° C. for 15 seconds and immediately thereafter cooled in an ice bath for sec. The samples were then treated by a stomacher for 120 Sec prior to the serial dilution and enumeration test. The viability of the bacteria was then determined and compared to that of the free bacteria exposed to the same conditions. The results are presented in Table 15 as follows:

TABLE 15
The effect of homogenization (prior to the pasteurization) on the viability of the bacteria.

| | | | Results | | |
|---|---|---|---|---|---|
| | | Bacteria/ | CFU per 1 g bacteria | | |
| Batch | | Microcapsules | | Pasteurization | Pasteurization |
| No. | Test Type | Quantity [g] | Control* | (15 sec at 72° C.) | (15 sec at 72° C.) |
| Free | Homogenizer | 5 | 3.2 × 10¹¹ | 2.75 × 10⁹ | |
| bacteria | | | | | |
| [04012021] | Homogenizer + | 0.01 | 3.3 × 10¹¹ | 3.6 × 10¹¹ | |
| | Pasteurization + | 5 | | 3.2 × 10¹¹ | |
| | Stomacher | | | | |
| [04012021] | Homogenizer + | 0.05 | 3.7 × 10¹¹ | 2.0 × 10⁸ | |
| 20% w/w | Pasteurization + | | | | |
| coating | Stomacher | | | | |
| [25012021] | Homogenizer + | 0.01 | 4.0 × 10¹¹ | 3.9 × 10¹¹ | |
| | Pasteurization + | 5 | | 3.8 × 10¹¹ | |
| | Stomacher | | | | |
| [25052021] | Homogenizer + | 5 | 1.1 × 10¹¹ | 4 × 10¹¹ | 1.75 × 10¹¹ |
| | Pasteurization + | | 1.7 × 10¹¹ | | |
| | Stomacher | | | | |
| [28022021] | Homogenizer + | 0.01 | 5.0 × 10¹¹ | 4.5 × 10¹¹ | |
| | Pasteurization + | 5 | | 3.3 × 10¹¹ | |
| | Stomacher | | | | |

*The control was the sample before both homogenization and simulated pasteurization processes

### Example 11—Stability in Milk at 42° C. Followed by Heating to 72-75° C.

### Test Procedure

First a certain quantity of microcapsules was accurately weighed and properly dispersed in 45 mL cow milk (3% fat) (40 mL for dispersion and additional 5 mL later for washing the residues left in the stomacher bag) in a stainless steel Turkish warmer coffee pot-Finjan at room temperature using a laboratory spoon for a few seconds. The resulting dispersion was heated to 42° C. for 240 minutes with no shaking (simulated fermentation process) followed by heating at 72° C. using a water bath (it took 70-75 seconds to reach the target temperature). The dispersion was then retained at 72° C. for 15 seconds (simulated pasteurization process) and immediately thereafter cooled in an ice bath for 30 sec. The samples were further treated first by a homogenizer for 30 sec at 5000 RPM followed by a stomacher (in a stomacher bag) for 120 Sec prior to the serial dilution and enumeration test. The viability of the bacteria was then determined and compared to that of the free bacteria exposed to the same conditions. The results are presented in Table 16 as follows:

TABLE 16
Viability of the bacteria after both simulated fermentation
and simulated pasteurization processes.

| | | Bacteria/ | | |
|---|---|---|---|---|
| | | Micro- | | |
| Batch | | capsules | | CFU per 1 g |
| No. | Test Type | Quantity [g] | Control* | bacteria |
| Free bacteria | Homogenizer | 5 | 3.2 × 10¹¹ | 2.75 × 10⁹ |
| [04012021] | Fermentation + | 0.01 | | 4 × 10¹⁰ |
| | Pasteurization + | | | |
| | Homogenizer + | | | |
| | Stomacher | | | |
| [25012021] | Fermentation + | 5 | | 4.2 × 10¹¹ |
| | Pasteurization + | | | |
| | Homogenizer + | | | |
| | Stomache | | | |
| [25052021] | Fermentation + | 5 | | 2.7 × 10¹¹ |
| | Pasteurization + | | | |
| | Homogenizer + | | | |
| | Stomache | | | |

*The control was the sample before both the simulated fermentation and simulated pasteurization processes.

### Example 12 Microencapsulated Bacteria for Powdered Infant Formula (PIF)

Resistance test for microencapsulated B. breve during reconstitution of PIF First, 1.8 g of B. breve-containing microcapsules (the w/w content of bacteria in the microcapsule was 20%) were properly mixed with 16.2 g of a commercial PIF (Nutrilon) to obtain a weight ratio of 1:10 of the microcapsules to PIF. The mixture was then added to a sterile glass infant bottle which contained 162 mL boiled water that had cooled to 70° C. (in total 180 g sample). The bottle was capped and after shaking for 10 seconds, to adequately disperse the powder mixture, was allowed to stand for different periods of time to complete the exposure time to 15-45 seconds (the exposure time to 70° C.). Thereafter, the glass bottle was cooled to 37° C. under a running tap water for 60-90 seconds. The PIF dispersion was then poured into a sterile stomacher bag and stomached for 2 minutes. Serial decimal dilutions were performed with TS+followed by plating using a pour-plate method with MRS agar as described above. The viable cell count was then performed to determine the CFU per 1 g of the bacteria considering the initial weight content of the bacteria in the final microcapsule.

Survival Assessment of the Bacteria

To evaluate the heat resistance and the survival of the microencapsulated bacteria the viability loss of the bacteria that occurred during the reconstitution of PIF at 70° C. (VLh) was calculated using the following equation:

VLh=Vc−Vh

Where:

Vc is the viable cell count right after microencapsulation, expressed as log (CFU per 1 g of bacteria), and Vh is the viable cell count after the exposure to 70° C. during the reconstitution of PIF in water for different periods of time, expressed as log (CFU per 1 g of bacteria).

The results are presented in Table 17 as follows:

TABLE 17
Viability of the microencapsulated bacteria after reconstitution
of PIF in water at 70° C. for different duration of times

| | | | Viability | | | |
|---|---|---|---|---|---|---|
| | Coating | % | [CFU/ g bacteria] | | | |
| Batch No. | Formula | Bacteria | Control** | 15 sec at 70° C. | 30 sec at 70° C. | 45 sec at 70° C. |
| [09012022] | 35% [92:6:2]* | 20 | 5.2 × 10¹¹ | 7.1 × 10¹⁰ | 6.0 × 10¹⁰ | 6.4 × 10¹⁰ |

| | |
|---|---|
| SA:ES200:Aerosil | Viability loss*** |
| | Log [CFU/ g bacteria] |

| | | | |
|---|---|---|---|
| — | 0.86 | 0.94 | 0.91 |

SA—Stearic acid
Aerosil ® is a trademark of Evonik (Germany) for hydrophilic fumed silica which was used as an anti-caking aid and flow aid.
Empure ® ES 200 is a specific trademark of Emsland Group (Germany) for a clean label pea starch (high amylose starch), with adjusted swelling and dissolving properties for the food industry; it was used as binding, thickening and shock-absorbing agent.
*The weight ratio between the components of the coating layer respectively
**The control was the microencapsulated bacteria right after the microencapsulation process before the reconstitution process of PIF in water at 70° C.
***Viability loss = VLh = Vc − Vh where Vc is the viable cell count right after the microencapsulation process, expressed as log (CFU per 1 g of bacteria), and Vh is the viable cell count after the exposure to 70° C. during the reconstitution of PIF in water for different periods of time, expressed as log (CFU per 1 g of bacteria)

The same test procedure was performed for the free bacteria to determine the survival of the bacteria (unencapsulated) during the reconstitution of PIF in water at 70° C. to evaluate the added value of the microencapsulation process and microcapsule structure in the resistance of the bacteria during the exposure to elevated temperatures. The results of the viability of the free bacteria before and after reconstitution in water at 70° C. are summarized in Table 18 which also presents the viability loss of the bacteria occurring during the reconstitution process.

TABLE 18
Viability of the free bacteria after reconstitution of PIF at 70° C. for different duration of times

| | Viability | | | | | | | | |
|---|---|---|---|---|---|---|---|---|---|
| | [CFU/g B. breve] | | | | | | | | |
| | | 10 Sec at | 20 Sec at | 30 Sec at | 40 Sec at | 50 Sec at | 60 Sec at | 180 Sec at | 300 Sec at |
| Batch No. | Control* | 70° C. | 70° C. | 70° C. | 70° C. | 70° C. | 70° C. | 70° C. | 70° C. |
| Uncoated | 6.0 × 10¹¹ | 2.4 × 10⁷ | 5.0 × 10⁷ | 1.0 × 10⁷ | 1.4 × 10⁷ | 1.0 × 10⁶ | 5.6 × 10⁶ | 1.6 × 10⁶ | 1.0 × 10⁵ |
| B. Breve | Viability loss** | | | | | | | | |
| | Log [CFU/g B. breve] | | | | | | | | |
| | — | 4.4 | 4.08 | 4.78 | 4.63 | 5.78 | 5.03 | 5.57 | 6.78 |

*The control was the free B. breve before the reconstitution process of PIF in water at 70° C.
**Viability loss = VLh = Vfc − Vfh where Vfc is the viable cell count of the free bacteria, expressed as log (CFU per 1 g of bacteria) before the exposure to 70° C., and Vfh is the viable cell count of the free bacteria after the exposure to 70° C. during the reconstitution of PIF in water for different periods of time, expressed as log (CFU per 1 g of bacteria)

Comparing the data emerging in Tables 17 and 18 one can clearly realize the meaningful protection that was endowed the bacteria during the reconstitution at elevated temperatures by the microencapsulation process and the microcapsule formulations according to the present invention.

### Example 13 Microencapsulated Probiotic Bacteria for Gummy/Jelly Candies

Microencapsulation Process

The microencapsulation process was performed by first creating the core followed by the coating process. The components used for the preparation of the microcapsules are summarized in Table 19.

The core was prepared by a melt granulation method; the cocoa butter melt was added gradually to the mixture of probiotic and maltodextrin. Alternatively, a melted liquid of the binder (e.g., stearic acid) was sprayed onto the powder mixture consisting of the bacteria and the filler to consolidate the particles to form agglomerates.

TABLE 19
The components of the microcapsules according to the present invention
for introducing probiotic bacteria into jelly candies

| Material Name | Use |
|---|---|
| Bifidobacterium Lactis, | Model bacteria for proof of concept |
| Lactobacillus Rhamnosus | |
| Maltodextrin | Filler |
| Stearic Acid | Binder/coating (hydrophobic component) |
| Arachidic acid | Coating (hydrophobic component) |
| Behenic acid | Coating (hydrophobic component) |
| ES 200 | Coating (edible water-soluble stress absorber) |
| Aerosil | Coating (anti-caking aid and flow aid) |
| Cocoa Butter | Binder/coating (hydrophobic component) |
| Hydroxypropyl cellulose | Outermost coating layer |
| L and J | |

Aerosil® is a trademark of Evonik (Germany) for hydrophilic fumed silica which was used as an anti-caking aid and flow aid.

Empure® ES 200 is a specific trademark of Emsland Group (Germany) for a clean label pea starch (high amylose starch), with adjusted swelling and dissolving properties for the food industry; it was used as binding, thickening and shock-absorbing agent.

For the coating process, first stearic acid was melted by heating a certain quantity of stearic acid (or arachidic acid or behenic acid) in proper container at 90° C.

After full melting, a certain quantity of ES 200 and Aerisol 200 powder was added into the melt while mixing until a uniform dispersion was achieved. The mixing was kept throughout the coating process.

The coating process took place by spraying the dispersion directly onto the resulting core from the previous stage. The coating process were carried out at Innojet Ventilus 2.5 (Romaco-Huttlin, Esteinen-Germany). The thickness of the layers was expressed by the % weight gain (WG) which was obtained upon the coating process in relative to the initial substrate's weight prior to the coating process according to the following equation:

$$\% WG = \frac{WG}{W 0} \times 100 = \frac{Wd - W0}{W 0} \times 100$$

Where Wd and W0 are respectively the weight of the substrate after and before coating process and WG is the weight gain.

In some cases, an outer layer consisting of a hydroxypropyl cellulose (for example, HPC L, HPC J, e.g., Klucel of Ashland) was utilized to amplify the protection capability of the microcapsules at temperature beyond the melting point of the hydrophobic component (stearic acid).

Survival Test

The resulting microcapsules were then added into jelly candies to assess the survival of the bacteria during the jelly production. For this purpose, first a pectin and chicory FOS (fructooligosaccharides) solution (100 g) was prepared at 80° C., then after cooling to 70-73° C., citric acid (0.08 g), flavors (0.02 g), and pigment (0.015 g) were added. After mixing all the components, the microcapsules (10.0 g) were added while mixing until a uniform solution was achieved. The resulting mixture was finally poured into a dog-bone shaped starch-based mold, using a plastic syringe, and after drying at room temperature for a few days, the jelly candies containing microencapsulated bacteria were obtained. The jelly candies were kept at 4° C. until the enumeration test was performed.

Enumeration Test Method

Mixing 10.0 g Jelly in 90.0 g TS+at room temperature until a full dissolution is obtained (40-45 minutes). After transferring into a sterile stomacher bag the suspension was stomached for 2.0 minutes. Decimal dilutions were finally performed with TS+followed by plating using a pour-plate method with MRS and TOS agars for Lactobacillus and Bifidobacterium respectively. The inoculated agar plates were then left on a cool even surface to solidify. After solidification, the plates were inverted and subsequently incubated anaerobically in a jar with the Anaerogen system, for 72±4 h at 37° C. The plates with 30-300 colonies were then used for calculating the colony-forming unit (CFU) per 1 g of the sample (CFU/g) as follows:

$$N = \frac{Σ C \times d}{V \times [ {n}_{1} + ( 0.1 \times {n}_{2} ) ]}$$

Where:
N=number of viable cells (CFU/g)
ΣC=sum of colonies on all plates containing 30-300 colonies
n1=number of the first plates (at dilution factor of d) containing 30-300 colonies
n₂=number of the second plates containing 30-300 colonies
d=dilution factor corresponding to the first plates showing 30-300 colonies
V=the volume poured into Petri dishes (V=1 mL)

Enumeration of microencapsulated B. breve To calculate the viability loss during all the process as compared to CFU/g of the free bacteria (control), the results were normalized to CFU per 1 g bacteria according to the following parameters.
Water content in jelly—20%
Sample taken for enumeration test—10 g of jelly
Microcapsules content in the sample −10%
Bacteria content in microcapsules—for B. lactis and L. rhamnosus— 18% except [22122020] which was 70%

The results of viability of microencapsulated bacteria based on different formulations according to the present invention as compared to the pure bacteria (unprotected) before and after exposure to the conditions of jelly candy production are presented in Table 20.

TABLE 20
Viability of the bacteria

| | | | | | | | Results | |
|---|---|---|---|---|---|---|---|---|
| | | Weight | | | Jelly | | | Viability |
| | | gain of | | Bacteria | production | Control | Viability | loss** |
| Microcapsules | Coating | coating | Bacteria | content | temperature | (CFU/g | (CFU/g | (log |
| Batch No. | Formula | % | Species | (% w/w) | ° C. | bacteria) | bacteria) | CFU/g) |
| Free bacteria | — | — | Bifidobacterium | 100 | 73-75 | 5.0 × 10¹¹ | 1.0 × 10¹⁰ | 1.7 |
| | | | lactis | | | | | |
| [22122020] | (92:6:2)* | 35% | Bifidobacterium | 70 | 73-75 | 2.1 × 10¹¹ | 1.1 × 10¹⁰ | 1.28 |
| | SA:ES200:Aerosil | Melt | lactis | | | | | |
| [17102021] | (86:12:2) | 35% | Bifidobacterium | 18 | 73-75 | 2.1 × 10¹¹ | 1.0 × 10¹¹ | 0.28 |
| | SA:ES200:Aerosil + | Melt + | lactis | | | | | |
| | HPC LF + | 5% | | | | | | |
| | HPC JF | HPC LF + | | | | | | |
| | | 5% | | | | | | |
| | | HPC JF | | | | | | |
| [17102021] | (86:12:2) | 35% | Bifidobacterium | 18 | 65-66 | 2.1 × 10¹¹ | 1.6 × 0¹¹ | 0.12 |
| | SA:ES200:Aerosil + | Melt + | lactis | | | | | |
| | HPC LF + | 5% | | | | | | |
| | HPC JF | HPC LF + | | | | | | |
| | | 5% | | | | | | |
| | | HPC JF | | | | | | |
| [17012022] | (92:6:2) | 35% | Bifidobacterium | 20 | 73-75 | 5.5 × 10¹¹ | | |
| | SA:ES:Aerosil | Melt | lactis | | | | | |
| [18012022]** | (92:6:2) | 50% | Bifidobacterium | 20 | 73-75 | 3.9 × 10¹¹ | 1.2 × 10¹¹*** | 0.51 |
| | SA:ES:Aerosil | Melt | lactis | | | | | |
| Free bacteria | | | Lactobacillus | 100 | | 3.3 × 10¹¹ | | |
| | | | rhamnosus | | | | | |
| [16112020] | (92:6:2) | 35% | Lactobacillus | 18 | 73-75 | 3.3 × 10¹¹ | 6.8 × 10¹⁰ | 0.69 |
| | SA:ES200:Aerosil | Melt | rhamnosus | | | | | |

Aerosil ® is a trademark of Evonik (Germany) for hydrophilic fumed silica which was used as an anti-caking aid and flow aid.
Empure ® ES 200 is a specific trademark of Emsland Group (Germany) for a clean label pea starch (high amylose starch), with adjusted swelling and dissolving properties for the food industry; it was used as binding, thickening and shock-absorbing agent.
*The weight ratio between the components of the coating layer respectively
**A cocoa butter melt was used as the binder
***The test was done after 14 days that was kept at 4° C.
a- The batch was prepared for feasibility of both coating formulation and the process (arachidic acid melting point = 75.4° C.)
b- The batch was prepared for feasibility of both coating formulation and process (behenic acid melting point = 80.0° C.)

It is to be noted that similar ratios have been tested with AA—Arachidic acid and BA-Behenic acid (namely, (92:6:2) AA:ES:Aerosil, (92:6:2) BA:ES:Aerosil) providing similar results as shown in table 20.

The results show that the microencapsulation with specific formulations according to the present invention protects the bacteria during the production of the jelly candies where the bacteria are exposed to harsh conditions involved in the process.

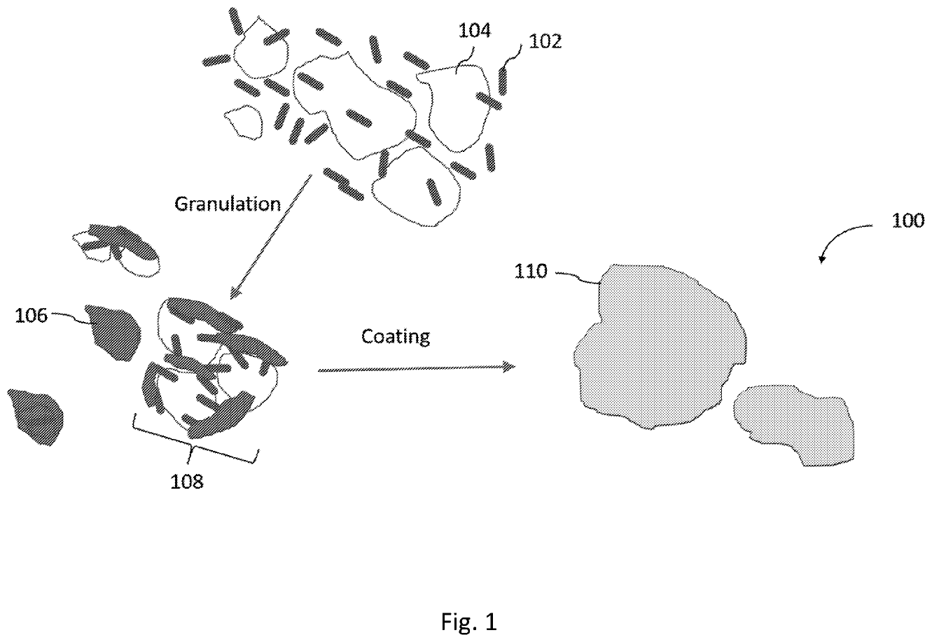

Reference is now made to FIG. 1 which is a schematic illustration of a process for the production of the probiotic granule 100 of the present invention, in accordance with some demonstrative embodiments.

According to some demonstrative embodiments, the process may include the absorbance of probiotic bacteria 102 onto Sugar particles 104.

According to some embodiments, sugar particles 104 may include for example oligosaccharide or polysaccharides particles.

According to some embodiments, bacteria 102 absorbed onto sugar particles 104 may be granulated with at least one edible solid fat, wax, phospholipid, or fatty acid 106 to result in probiotic particles 108.

According to some embodiments, probiotic particles 108 may then be coated with at least one edible hydrophobic solid component 110 to provide probiotic granule 100 of the present invention.

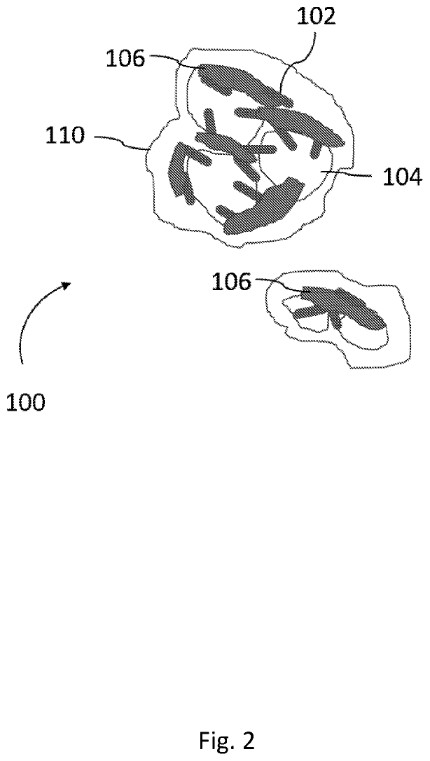

Reference is now made to FIG. 2 which is an illustration of a cross section plane of probiotic granule 100 of the present invention, in accordance with some demonstrative embodiments.

FIG. 2 shows probiotic bacteria 102 absorbed onto sugar particles 104 and granulated with at least one edible solid fat, wax, phospholipid or fatty acid 106, whereas the resulting particle is coated with at least one edible hydrophobic solid component 110.

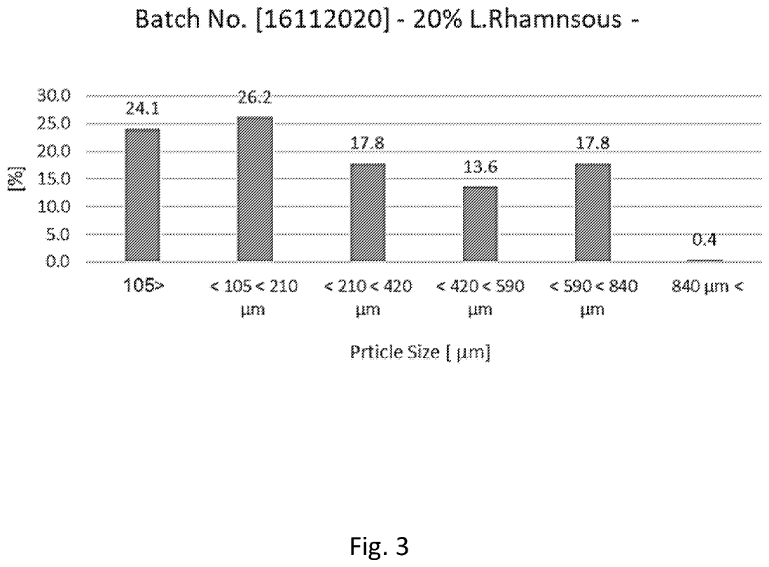

FIG. 3 is a graph depicting particle size of batch No. [16112020]—20% Lactobacillus Rhamnsous LGG.

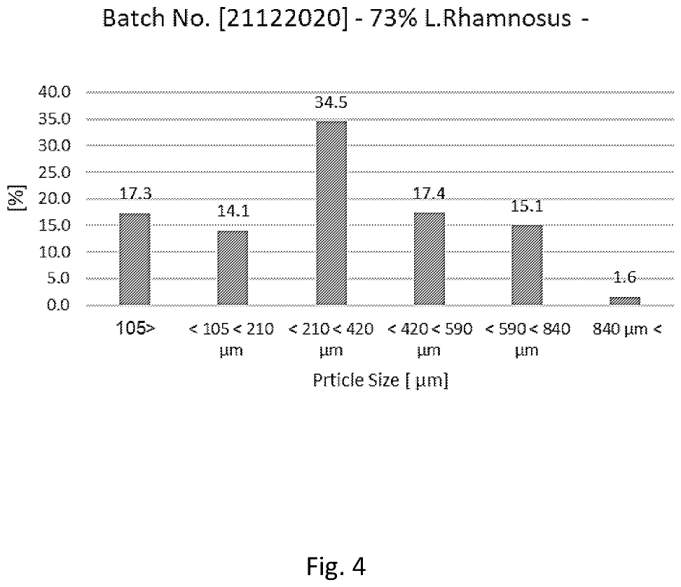

FIG. 4 is a graph depicting particle size of batch No. [21122020]—73% Lactobacillus Rhamnsous

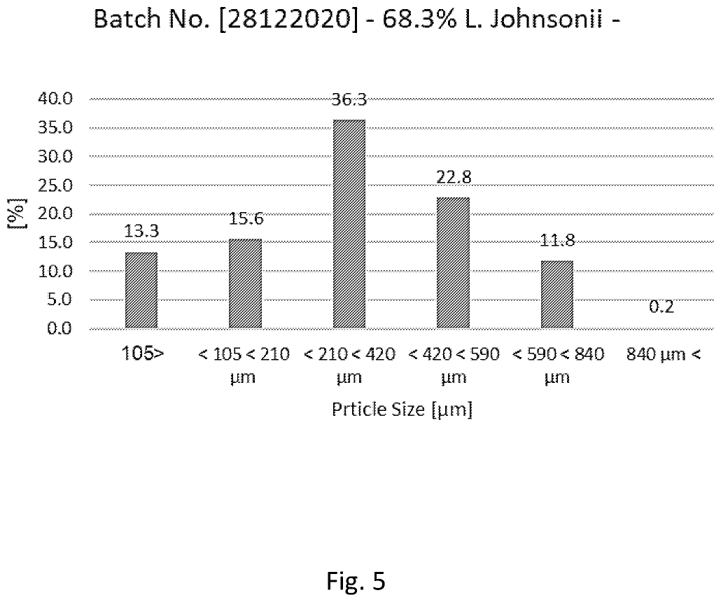

FIG. 5 is a graph depicting particle size of batch No. [28122020]—68.3% Lactobacillus Johnsonii

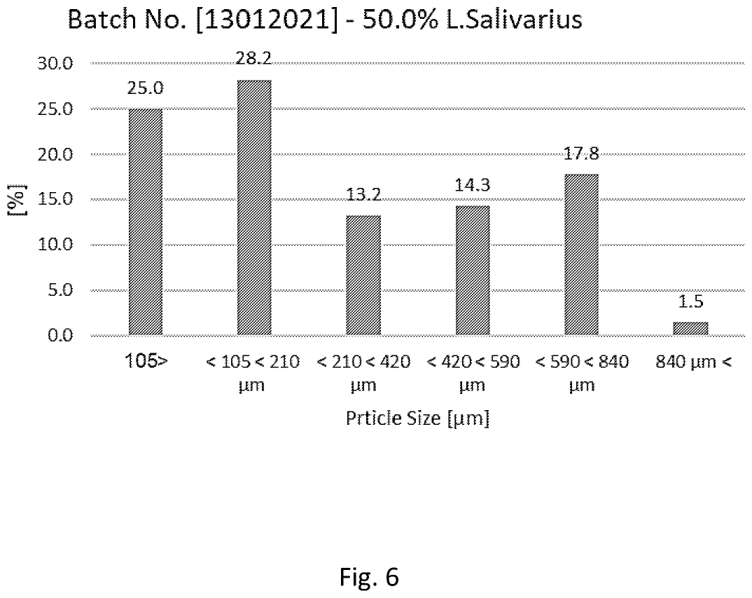

FIG. 6 is a graph depicting particle size of batch No. [13012021]—50.0% Lactobacillus Salivarius

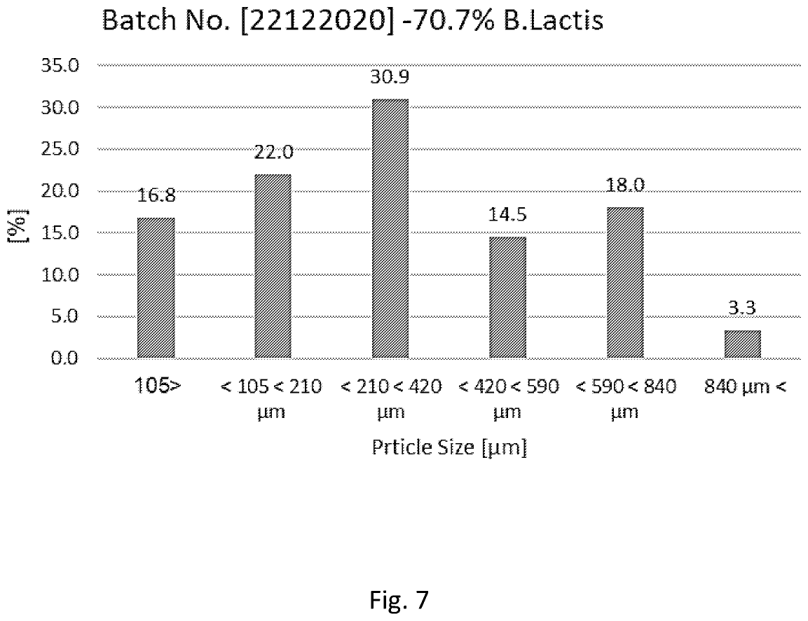

FIG. 7 is a graph depicting particle size of batch No. [22122020]-70.7% Bifidobacterium Lactis

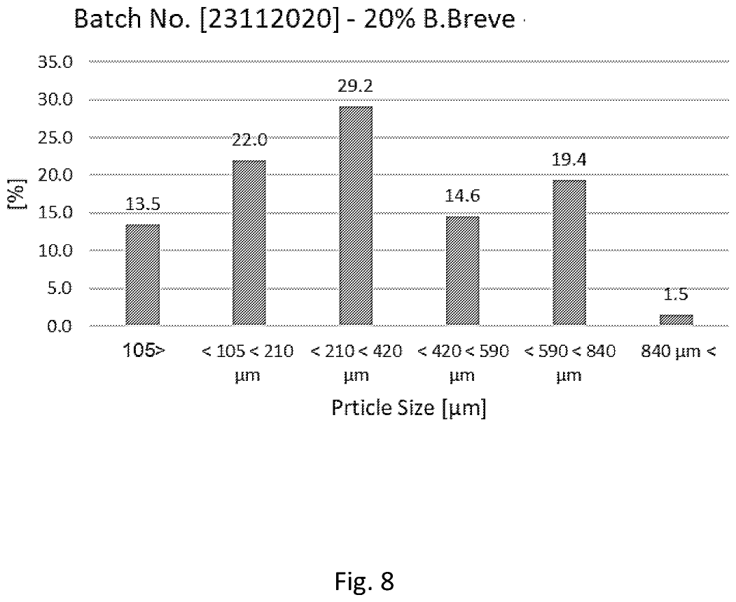

FIG. 8 is a graph depicting particle size of batch No. [23112020]—20% Bifidobacterium Breve

While this invention has been described in terms of some specific examples, many modifications and variations are possible. It is therefore understood that within the scope of the appended claims, the invention may be realized otherwise than as specifically described.

1. A probiotic granule comprising:
a core comprising probiotic bacteria; and
a continuous solid dispersion coating layer coating said core comprising
at least one an edible hydrophobic solid component such as fat, wax, phospholipid or a fatty acid having a melting point higher than 30° C.;
at least one edible water-soluble polymeric stress absorber, dispersed within said hydrophobic solid component.

2. The granule of claim 1, wherein said at least one edible water-soluble polymeric stress absorber is in the form of particles having a mean average size of 150 μm or less.

3. The granule of claim 1, wherein said at least one edible water-soluble polymeric stress absorber has an impact force of 100-1000 N/m.

4. The granule of claim 1, wherein said at least one edible water-soluble polymeric stress absorber is selected from the group including Starches, Gums, Cellulose ethers and Vinyl polymers.

5. The granule of claim 1, wherein said at least one edible water-soluble polymeric stress absorber is high amylose starch.

6. The granule of claim 4, wherein a weight percentage of said edible water-soluble polymeric stress absorber in said solid dispersion coating layer is between 1% and 20% w/w.

7. The granule of claim 1, wherein said hydrophobic solid component has a melting point higher than 30° C. and below 80° C.

8. The granule of claim 7, wherein said hydrophobic solid component is stearic acid.

9. The granule of claim 1, wherein said solid dispersion coating layer is in an amount of 10-50% weight gain (WG).

10. The granule of claim 1, further comprising a second layer, wherein said second layer is an enteric layer comprising an edible polymer selected from the group including pH-sensitive polymers, for example, acid phthalate of carbohydrates, amylose acetate phthalate, cellulose acetate phthalate (CAP), other cellulose ester phthalates, cellulose ether phthalates, hydroxypropylcellulose phthalate (HP CP), hydroxypropylethylcellulose phthalate (HPECP), hydroxyproplymethylcellulose phthalate (HPMCP), hydroxyproplymethylcellulose acetate succinate (HPMCAS), methylcellulose phthalate (MCP), polyvinyl acetate phthalate (PVAcP), polyvinyl acetate hydrogen phthalate, sodium CAP, starch acid phthalate, cellulose acetate trimellitate (CAT), styrene and maleic acid copolymers, styrene-maleic acid dibutyl phthalate copolymer, styrene-maleic acid/polyvinylacetate phthalate copolymer, polyacrylic acid derivatives such as acrylic acid and acrylic ester copolymers, polymethacrylic acid and esters thereof, polyacrylic and methacrylic acid copolymers, polyacrylic acid derivatives such as particularly copolymers comprising acrylic acid and at least one acrylic acid ester, Eudragit STM (poly(methacrylic acid, methyl methacrylate)1:2); Eudragit LTM which is an anionic polymer synthesized from methacrylic acid and methacrylic acid methyl ester), Eudragit L100TM (poly(methacrylic acid, methyl methacrylate)1:1); Eudragit L30DTM, (poly(methacrylic acid, ethyl acrylate)1:1); and Eudragit L100-55TM (poly(methacrylic acid, ethyl acrylate)1:1), polymethyl methacrylate blended with acrylic acid and acrylic ester copolymers, alginic acid and alginates such as ammonia alginate, sodium, potassium, magnesium or calcium alginate or a combination thereof.

11. A process for the production of a probiotic granule, wherein the process includes:
forming a core using a hot melt process comprising:
I. Mixing probiotic bacteria with sugar particles
II. Melting at least one edible solid fat, wax or fatty acid;
III. Spraying the melt of said at least one edible solid fat, wax or fatty acid onto the mixture of said probiotic bacteria with sugar particles to form a solid core;
coating said core, comprising:
I. Melting at least one an edible hydrophobic solid component having a melting point higher than 30° C. to form a hydrophobic film;
II. Adding and mixing at least one edible water-soluble polymeric stress absorber with the melt of said at least one an edible hydrophobic solid component to yield a melt of hydrophobic mixture;
III. Spraying the melt of the hydrophobic mixture onto the core to provide a probiotic granule.

12. The process of claim 11, wherein said process further includes coating said probiotic granule with a second layer, comprising an edible enteric polymer to further additional protection wherein said second layer comprises a polymer selected from the group including HIPC or an enteric coating polymer.

13. Use of the granule of claim 1 in the preparation of a food product

14. A food product comprising the granule of claim 1, selected from the group including grains and grain products, dairy products, and sweets.

15. The food product of claim 14, wherein said sweets are selected from the group including candies, jelly candies, gummies, lollipops, chewing gums, caramels, chocolate candies and chocolate.
